SEQUENCE LISTING (8 sequences)

SEQ ID NO: 1 - DNA, 3254 bp, Homo sapiens
caccgtccgcagcccgagcgccccggagccgcaggcgccgccgcgcagagacgccgcggctgcgactaggcgcgcccagccgcacgtggcggacccgcccccaggcccgcagtgtcctggaccccgcaggcctccgctctcctgtcctcggccccgtccccagggccgcgatgagcttcctgagccgacagcagccgccgccaccccgccgcgccggggcggcctgcaccttgcggcagaagctgatcttctcgccctgcagcgactgtgaggaggaggaagaagaggaggaggaggagggcagcggccacagcaccggggaggactcggcctttcaagagcccgactcgccgctgccgcccgagcggagccccacggagcccgggcccgagcgccgccgctcgcccgggccggcccccggcagccccggcgagctggaggaggacctgttgctgcccggcgcctgcccgggcgcggacgaggcgggcggtggggcggagggcgactcgtgggaggaggagggcttcggctcctcgtcgccggtcaagtcgccggcggccccctacttcctgggtagctctttctcgccggtgcgctgcggcggcccaggagatgcgtcgccgcggggttgcggggcgcgccgggcgggcgaaggccgccgctcgccgcggccggaccacccgggcaccccgccacacaagaccttccgcaagctgcgactcttcgacaccccgcacacgcccaagagtttgctctccaaagctcggggaattgattccagctctgttaaactccggggtagttctctcttcatggatacagaaaaatcaggaaaaagggaatttgatgtgcgacagactcctcaagtgaatattaatccttttactccggattctttgttgcttcattcctcaggacagtgtcgtcgtagaaagagaacgtattggaatgattcctgtggtgaagacatggaagccagtgattatgagcttgaagatgaaacaagacctgctaagagaattacaattactgaaagcaatatgaagtcccggtatacaacagaatttcatgagctagagaaaatcggctctggagaatttggttctgtatttaagtgtgtgaagaggctggatggatgcatttatgccattaagcgatcaaaaaagccattggcgggctctgttgatgagcagaacgctttgagagaagtatatgctcatgcagtgcttggacagcattctcatgtagttcgatatttctctgcgtgggcagaagatgatcatatgcttatacagaatgaatattgtaatggtggaagtttagctgatgctataagtgaaaactacagaatcatgagttactttaaagaagcagagttgaaggatctccttttgcaagttggccgaggcttgaggtatattcattcaatgtctttggttcacatggatataaaacctagtaatattttcatatctcgaacctcaatcccaaatgctgcctctgaagaaggagacgaagatgattgggcatccaacaaagttatgtttaaaataggtgatcttgggcatgtaacaaggatctccagtccacaagttgaggagggcgatagtcgttttcttgcaaatgaagttttacaggagaattatacccatctaccaaaagcagatatttttgcgcttgccctcacagtggtatgtgctgctggtgctgaacctcttccgagaaatggagatcaatggcatgaaatcagacagggtagattacctcggataccacaagtgctttcccaagaatttacagagttgctaaaagttatgattcatccagatccagagagaagaccttcagcaatggcactggtaaagcattcagtattgctgtccgcttctagaaagagtgcagaacaattacgaatagaattgaatgccgaaaagttcaaaaattcacttttacaaaaagaactcaagaaagcacagatggcaaaagctgcagctgaggaaagagcactcttcactgaccggatggccactaggtccaccacccagagtaatagaacatctcgacttattggaaagaaaatgaaccgctctgtcagccttactatatactgagctactcctttcccacctccccctgaacactgtgacaagaggaagctaggttgaaatcactgatagaatccagtttgcaattactttctcgattggtgtcagtagttttactgattaggacttttattgtgaattacagttgaaagctgtattttgatgattgctatgtcaggctttcatctaatcttaccagtctgtcttctgtaggatgtgtcactgttggatgttacaccagcctttccagggttaaccactgtggtggtgtgctgcttatagtttgctgttgcattgtaataaaaggtgtctttccctgtagtgacctgtaaaaagtactcaagggctttattacagacataccctccctttgaaaagggacatgctaaaagactcattactactcagccttcaatgtacctgtgtgtccatcttatatttcttttttttttaattgtgaattagacttgtatatcccactgggagcactttgtaggcattgcatgaaccatgggatgatgattctgtggaggtattgccttgtgaatttgctgctattttagttttgtctttgctgtaaacttgtagcattaaacaatcattgttgttaataggtcttctttttgaaacaattatgtgaaatgtatagctgcttttgatggaaagcagctatttgcctttttttttttcctttgaactttgaagctagtgcattggaaaaatgcaccctttccctcctttggaatgctgtattaatgtagtataataattactggttttgtaacttgttctggtaatgtccttcccggactctttttaaatgtctccccctaagttttatacttgattgtattattagtctgtttttaaatgttttgcccggtttttctcttcaatatttgtgtatataaaccgatcttcgtgatactgtacatagctgtttgaaatgccagaatgacttctgacattccaagtttttcacaaaatatattttatctgtgattagccatttgactaataatactggctaacagatgttgaaaaaaattgtctgttttctcattaattttggtctaaaacatgtttgcacttgtctttgacttgtgttttattaacattgattggcatattaaaagtcactctgagctt

SEQ ID NO: 2 - protein, 646 aa, Homo sapiens [sequence omitted]

SEQ ID NO: 3 - DNA, 36 bp, Homo sapiens
cagggcgccatgggaatgaagtcccggtatacaaca

SEQ ID NO: 4 - protein, 12 aa, Homo sapiens
QGAMGMKSRYTT

SEQ ID NO: 5 - DNA, 36 bp, Homo sapiens
cctttgaattcactttctagaagcggacagcaatac

SEQ ID NO: 6 - protein, 8 aa, Homo sapiens
KRSASLLV

SEQ ID NO: 7 - protein, 312 aa, Homo sapiens
MSYYHHHHHHDYDIPTTENLYFQGAMGMKSRYTTEFHELEKIGSGEFGSVFKCVKRLDGCIYAIKRSKKPLAGSVDEQNALREVYAHAVLGQHSHVVRYFSAWAEDDHMLIQNEYCNGGSLADAISENYRIMSYFKEAELKDLLLQVGRGLRYIHSMSLVHMDIKPSNIFISRTSIPNAASEEGDEDDWASNKVMFKIGDLGHVTRISSPQVEEGDSRFLANEVLQENYTHLPKADIFALALTVVCAAGAEPLPRNGDQWHEIRQGRLPRIPQVLSQEFTELLKVMIHPDPERRPSAMALVKHSVLLSASRK

SEQ ID NO: 8 - protein, 289 aa, Homo sapiens
GAMGMKSRYTTEFHELEKIGSGEFGSVFKCVKRLDGCIYAIKRSKKPLAGSVDEQNALREVYAHAVLGQHSHVVRYFSAWAEDDHMLIQNEYCNGGSLADAISENYRIMSYFKEAELKDLLLQVGRGLRYIHSMSLVHMDIKPSNIFISRTSIPNAASEEGDEDDWASNKVMFKIGDLGHVTRISSPQVEEGDSRFLANEVLQENYTHLPKADIFALALTVVCAAGAEPLPRNGDQWHEIRQGRLPRIPQVLSQEFTELLKVMIHPDPERRPSAMALVKHSVLLSASRK

(19) United States
(12) Patent Application Publication
(10) Pub. No.: US 2005/0037476 A1
(43) Pub. Date: Feb. 17, 2005

(54) Modified Wee1, crystals of peptide: inhibitor complexes containing such modified Wee1, and methods of use thereof

(75) Inventors: Edward Neill Baker, Takapuna (NZ); Richard John Booth, Ann Arbor, MI (US); Alan J. Kraker, Ann Arbor, MI (US); Daniel Fred Ortwine, Saline, MI (US); James Michael Jeremy Dickson, Beachhaven (NZ); Ivan Ivanovic, Ellerslie (NZ); Christopher John Squire, Devonport (NZ)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 10/837,652

(22) Filed: May 4, 2004

Related U.S. Application Data
(60) Provisional application No. 60/473,887, filed on May 29, 2003.

(51) Int. Cl. G06F 19/00; G01N 33/48; G01N 33/50; C12N 9/99; C12N 9/22

(52) U.S. Cl. 435/184; 435/199; 702/19

(57) ABSTRACT

Modified Wee1 peptides, polynucleotides encoding those peptides, and methods for purifying the peptides and crystallizing them as peptide: inhibitor complexes have been discovered. The three-dimensional structure of Wee1, including the ATP substrate binding site, and uses of this information in the design and screening of compounds that may associate with Wee1, or peptides structurally related thereto, have also been discovered.

### FIELD OF THE INVENTION

The present invention generally relates to modified forms of a Wee1 peptide, crystals of modified Wee1 peptide: inhibitor complexes, methods for producing those crystals, three-dimensional structural information derived from crystallographic data, and to methods for using that structural information in the design of potential Wee1 inhibitor compounds.

### Reference to Tables 1-5 Submitted on Compact Disc

The three-dimensional atomic coordinates in the following Tables 1-5 are submitted herewith on duplicate compact discs. The material on the compact discs is incorporated by reference herein. The files on the compact discs, containing the atomic coordinates of Tables 1-5, are labeled Table 1, Table 2, Table 3, Table 4, and Table 5, respectively.

Table 1. Three-Dimensional Atomic Coordinates From the X-Ray Crystal Structure of a Wee1₂₉₁₋₅₇₅: 9-Hydroxy-4-phenyl-6H-pyrrolo[3,4-c]carbazole-1,3-dione Complex with Mg (207 KB);

Table 2. Three-Dimensional Atomic Coordinates From the X-Ray Crystal Structure of a Wee1₂₉₁₋₅₇₅: 4-(2-Chloro-phenyl)-8-(3-dimethylamino-propoxy)-9-hydroxy-6-methyl-6H-pyrrolo[3,4-c]carbazole-1,3-dione Complex (397 KB);

Table 3. Three-Dimensional Atomic Coordinates From the X-Ray Crystal Structure of a Wee1₂₉₁₋₅₇₅: 3-(9-Hydroxy-1,3-dioxo-4-phenyl-2,3-dihydro-1 H-pyrrolo[3,4-c]carbazol-6-yl)-propionic acid Complex (198 KB);

Table 4. Three-Dimensional Atomic Coordinates From the X-Ray Crystal Structure of a Wee1₂₉₁₋₅₇₅: 9-Hydroxy-6-(3-hydroxy-propyl)-4-(2-methoxy-phenyl)-6H-pyrrolo[3,4-c]carbazole-1,3-dione Complex (187 KB); and

Table 5. Three-Dimensional Atomic Coordinates From the X-Ray Crystal Structure of a Wee1₂₉₁₋₅₇₅: [8-(3-Amino-pyrrolidine-1-carbonyl)-4-(2-chloro-phenyl)-6-methyl-6H-pyrrolo[3,4-c]carbazole-1,3-dione Complex (205 KB).

### BACKGROUND OF THE INVENTION

Cell cycle kinases are naturally occurring enzymes involved in regulation of the cell cycle. Some of these kinases are responsible for inhibiting the cell's normal progression through to cell division, while others are normally active in promoting the progression of cells through the cell cycle leading to cell division. Increased activity or temporally abnormal activation of these kinases has been shown to result in development of tumors and other proliferative disorders such as potential disease states that involve diverse cellular processes including apoptosis, differentiation, angiogenesis and inflammation. As a result, inhibitors of cell cycle kinases are expected to be effective in the treatment of a wide variety of diseases, including, but not limited to cancer, psoriasis, arthritis, septic shock, viral infections and cardiovascular disease.

The proper orchestration of the steps required for orderly progression through the cycle that describes cell division makes use of a number of signaling pathways within cells. Many of these pathways utilize protein kinases to effect the transmission of these crucial signals at the appropriate time and intracellular location. One of the protein kinases involved is a tyrosine specific kinase, Wee1, that has as its substrate another kinase complex called Cdc2/cyclinB. Other known Wee kinases include, for example, Myt1 kinase, Wee1b kinase (Makoto N., et al, Genes to Cells 5: 839-847 (2000)), and Wee2 kinase (Leise, W. F., & Mueller, P. R., Developmental Biology 249: 156-173 (2002)).

The kinase activity of Cdc2/cyclinB is absolutely required for cells to progress through the G₂ stage of the cell division cycle to the M (or mitotic) phase where two daughter cells are formed from the division of the parent cell. Wee1 kinase is a regulatory kinase that has Cdc2/cyclinB as its substrate and when Wee1 kinase is active, it phosphorylates a specific tyrosine (Tyr15) on Cdc2 that causes an inactivation of the Cdc2/cyclinB complex, which in turn results in a pause or checkpoint in the cell cycle at the G₂ and M transition. Under normal circumstances, as cells are progressing through the cell cycle, the Cdc2/cyclinB complex is assembled in late S phase and through G₂. Wee1 kinase is normally active and thus phosphorylates the Cdc2/cyclinB complex until the end of G₂ when all of the necessary components have been synthesized for the entry of cells into M phase, whereupon Wee1 activity diminishes, a phosphatase removes the inhibitory phosphorylation from Tyr15 of the Cdc2/cyclinB complex, and the complex therefore becomes activated and cells move into M phase where the replicated DNA is divided and the daughter cells are formed. Inhibition of Wee1 kinase results in no inhibitory phosphorylation of Tyr15 on the Cdc2/cyclinB complex and the potentially inappropriate and premature entry of the cell into mitosis. See, Watanabe, N. et al., “Regulation of the Human WEE1Hu CKD Tyrosine 15-Kinase During the Cell Cycle,” The EMBO Journal, 14:1878-1891 (1995).

In addition to regulation of the transition of cells between the different phases of the cell cycle under normal conditions, the cell cycle transitions are regulated in response to damage to DNA, presumably giving cells opportunities either to repair potentially genotoxic DNA damage before replication using a damaged DNA template or to permanently exit the cell cycle and die. Inhibition of Wee1 kinase in the presence of DNA damaged by conventional DNA-directed chemotherapeutic agents or by radiation presents an opportunity to utilize cellular regulatory pathways to inappropriately and prematurely cause cells less likely to survive to progress into M phase and further divide since the commitment to M phase was made in the presence of potentially catastrophically damaged DNA (Kraker et al., Ann. Rep. Med. Chem. 34:247-256, (1999)).

One such means of mediating the cell cycle pathway is to identify inhibitors/antagonists to the Wee1 kinase. Such identification has heretofore relied on serendipity and/or systematic screening of large numbers of natural and synthetic compounds. A superior method of drug development relies on structure based drug design. In this case, the three dimensional structure of a peptide-inhibitor complex is determined and potential inhibitors/antagonists are screened, modified, and/or designed with the aid of computer modeling (Bugg et al., Scientific American, 92-98 (December 1993); West et al., TIPS, 16:67-74 (1995); Dunbrack et al., Folding & Design, 2:27-42 (1997)). However, heretofore the three dimensional structure of the Wee1 peptide, including its ATP substrate binding site, has remained unknown, essentially because no peptide crystals have been produced of sufficient quality to allow the required X-ray crystallographic data to be obtained. Therefore, there is presently a need for obtaining a form of the Wee1 peptide that can be crystallized with an inhibitor to form a crystal with sufficient quality to allow such crystallographic data to be obtained. Further, there is a need for such crystals. There is also a need for the determination of the three dimensional structure of the Wee1 peptide: inhibitor complex based on the X-ray diffraction of said crystals. Finally, there is a need for procedures for related structure-based drug design and/or screening based on the crystallographic data.

### SUMMARY OF THE INVENTION

The present invention provides modified Wee1 peptide amino acid sequences, and methods for producing such modified Wee1 peptide sequences. In one embodiment, the Wee1 peptides may be modified by omitting a significant portion of the NH₂-terminal region and the COOH-terminal region of the Wee1 peptide(s) (hereinafter referred to as “modified” Wee1 peptide(s)). The modified Wee1 peptides preferably lack (1) at most about 290 amino acid residues from the NH₂-terminal region of the full length Wee1 peptide; and (2) at most about 70 amino acid residues from the COOH-terminal region of the full length Wee1 peptide. Most preferably, the modified Wee1 peptides lack 290 amino acid residues from the NH₂-terminal region of the full length Wee1 peptide, and 70 amino acid residues from the COOH-terminal region of the full length Wee1 peptide. The modified Wee1 peptides may also optionally include a histidine oligomer at NH₂-terminal end.

The invention also provides peptides that are defined by the three-dimensional atomic coordinates of the modified Wee1 peptides as set forth in any one of Tables 1-5, or by three-dimensional atomic coordinates having a root mean square deviation of most preferably not more than about 1.25 Å away from the core carbon-alpha atoms of the modified Wee1 peptides as set forth in any one of Tables 1-5.

Additionally, the invention provides peptides that comprise a Wee1 peptide ATP substrate binding site or Wee1-like peptide ATP substrate binding site, as defined below.

The invention further provides isolated, purified polynucleotides that encode the modified Wee1 peptide sequences. The polynucleotide may be natural or recombinant.

The invention further provides expression vectors for producing the modified Wee1 peptides in a host cell. The host cell may be stably transformed and transfected with a polynucleotide encoding the modified Wee1 peptides, or a fragment thereof or an analog thereof, in a manner allowing the expression of the modified Wee1 peptides. The present invention additionally includes a cell transfected or transformed with an expression vector of the present invention. The present invention also includes methods for expressing a modified Wee1 peptide comprising culturing a cell that expresses the modified peptide in an appropriate cell culture medium under conditions that provide for expression of the peptide by the cell.

Methods of purifying the modified Wee1 peptides of the invention from a cell culture containing a modified peptide and contaminant proteins other than the peptide is also provided. The method comprises subjecting the cell culture to host cell lysis, immobilized metal affinity chromatography, removal of a his-tag or other suitable tags and linker sequences at the N- or C-terminus of the modified Wee1 peptide, and size exclusion chromatography. Preferably, the method comprises subjecting the cell culture to mechanical lysis, cobalt metal affinity chromatography, histidine tag cleavage, dialysis, size exclusion chromatography, and concentration. The method may further comprise utilizing one or more suitable buffer(s), preferably having a pH of about 7 to about 8, an inorganic salt, a reducing agent, and an organic compound. Preferably, a stabilizing agent is used in the method, such as, for example, about 2% to about 5% glycerol, to stabilize the protein in solution at various stages of the purification, preventing protein precipitation.

The invention also provides methods for producing inhibitor bound complexes of modified Wee1 peptide, and for producing crystals of the modified Wee1 peptide: inhibitor complexes. The methods for producing a Wee1 peptide: inhibitor crystalline complex comprise contacting a purified, modified Wee1 peptide with an inhibitor to form a Wee1: inhibitor binary complex; and adding the solution of the binary complex to a crystallization solution to form a Wee 1 peptide: inhibitor crystal. Preferably, the Wee1 peptide is contacted with the inhibitor in the presence of one or more of a buffering agent, a reducing agent, a source of ionic strength, an organic agent, and a metal cation chelating agent. Additionally, the binary complex is preferably added to a crystallization solution comprising one or more of a source of ionic strength and a buffering agent. In one embodiment, a method of producing crystals comprises contacting about 4 mg/ml to about 8 mg/ml purified Wee1 peptide with about 1 to about 2 molar excess of an suitable Wee1 inhibitor; and adding the resultant binary Wee1 peptide: inhibitor complex solution to a crystallization solution at a ratio of about 1:1. The crystallization solution preferably comprises a HEPES buffer at a pH of about 5.5 to about 9.0, and NaCl at a concentration of about 3.0 M to about 5.0 M. Preferably, the method for producing crystals comprises subjecting the binary Wee1 peptide: inhibitor complex to a hanging drop method of vapor diffusion to produce suitable crystals.

The invention also provides crystalline structures of the modified Wee1 peptides of the invention, or a structurally related peptide, in complex with an inhibitor, from which crystallographic structural information may be obtained. The crystals preferably have a resolution greater that 5.0 Å, more preferably greater than 2.5 Å. The crystalline structures of the modified Wee1 peptide complexes are the first reported of the Wee1 peptide or any of the related Wee1 peptide family. Preferably, the inhibitor is an inhibitor selected from the group consisting of:
9-Hydroxy-4-phenyl-6H-pyrrolo[3,4-c]carbazole-1,3-dione;
4-(2-Chloro-phenyl)-8-(3-dimethylamino-propoxy)-9-hydroxy-6-methyl-6H-pyrrolo[3,4-c]carbazole-1,3-dione;
3-(9-Hydroxy-1,3-dioxo-4-phenyl-2,3-dihydro-1 H-pyrrolo[3,4-c]carbazol-6-yl)-propionic acid;
9-Hydroxy-6-(3-hydroxy-propyl)-4-(2-methoxy-phenyl)-6H-pyrrolo[3,4-c]carbazole-1,3-dione; and
8-(3-Amino-pyrrolidine-1-carbonyl)-4-(2-chloro-phenyl)-6-methyl-6H-pyrrolo[3,4-c]carbazole-1,3-dione.

The invention also provides three-dimensional atomic coordinates of the modified Wee1 peptide: inhibitor complexes, as set forth in Tables 1-5, or a related set of atomic coordinates having a root mean square deviation of preferably not more than about 1.25 Å away from the core C alpha atoms of the three-dimensional atomic coordinates as set forth in any one of Tables 1-5. The atomic coordinates reflect the three-dimensional structure of the modified Wee1 peptide binary complexes and illustrate to atomic resolution the chemical environment around the ATP substrate binding site where Wee1 inhibitors bind.

Particularly, it has been discovered that Wee1 comprises an ATP substrate binding site that is defined by the three-dimensional atomic coordinates of the following amino acid residues within about 4 Å of an inhibitor located in the ATP substrate binding site: I305, G306, V313, A326, K328, E346, V360, I374, N376, E377, Y378, C379, N380, G382, F433, D463 of SEQ ID NO: 2, or a conservatively substituted variant thereof. Additionally, it has been discovered that Wee1 comprises an ATP substrate binding site that is defined by the three-dimensional atomic coordinates of the following amino acid residues within about 5 Å of an inhibitor located in the ATP substrate binding site: I305, G306, V313, A326, K328, E346, V360, I374, N376, E377, Y378, C379, N380, G381, G382, S383, D386, N431, F433, G462, D463, L464, G465 of SEQ ID NO: 2, or a conservatively substituted variant thereof.

It has also been discovered that the Wee1 peptide ATP substrate binding site is defined by the following amino acid residues: Y378, I305, V313, A326, G382, and F433 of SEQ ID NO: 2, or a conservatively substituted variant thereof, that make hydrophobic interactions with inhibitors having, for example, a carbazole or similar ring system. Further, it has been discovered that other amino acid residues, such as, C379, G377, and N376 of SEQ ID NO: 2, or a conservatively substituted variant thereof, make hydrogen bonding interactions with, for example, 9-hydroxy, 1-carbonyl and 2-NH substituents of inhibitors having, for example, a carbazole or similar ring system. These discoveries can be used, for example, in the design of Wee1 kinase inhibitors.

Additionally, it has been discovered that Wee1 comprises an ATP substrate binding site defined by the following amino acid residues: V313, K328, E346, D463, N376, and I374 of SEQ ID NO: 2, or a conservatively substituted variant thereof. Optionally, the binding site may additionally be defined by tightly bound water molecules, and/or a bound Magnesium atom and its associated waters. Observations that the amino acid residues forming this site will accommodate groups such as phenyl rings (unsubstituted or substituted) or other groups of similar size, can be used in, for example, the design of Wee1 kinase inhibitors.

Accordingly, another aspect of the invention is an isolated and purified polypeptide consisting of an ATP substrate binding site defined by

(a) the three-dimensional atomic coordinates of the following amino acid residues within about 4 Å of an inhibitor located in the binding site: I305, G306, V313, A326, K328, E346, V360, I374, N376, E377, Y378, C379, N380, G382, F433, D463 of SEQ ID NO: 2, or a conservatively substituted variant thereof;

(b) the three-dimensional atomic coordinates of the following amino acid residues within about 5 Å of an inhibitor located in the binding site: I305, G306, V313, A326, K328, E346, V360, I374, N376, E377, Y378, C379, N380, G381, G382, S383, D386, N431, F433, G462, D463, L464, G465 of SEQ ID NO: 2, or a conservatively substituted variant thereof;

(c) an ATP substrate binding site defined by the following amino acid residues: I305, V313, A326, N376, E377, Y378, C379, G382, and F433 of SEQ ID NO: 2, or a conservatively substituted variant thereof;

(d) an ATP substrate binding site defined by the following amino acid residues: V313, K328, E346, D463, N376, and I374 of SEQ ID NO: 2, or a conservatively substituted variant thereof;

(e) an ATP substrate binding site defined by the following amino acid residues: V313, K328, E346, D463, N376, and I374 of SEQ ID NO: 2, or a conservatively substituted variant thereof, and a bound magnesium ion and its associated waters; and

(f) an ATP substrate binding site defined by the following amino acid residues: V313, K328, E346, D463, N376, and I374 of SEQ ID NO: 2, or a conservatively substituted variant thereof, and tightly bound water molecules.

The invention also provides a Wee1 peptide ATP substrate binding site or a Wee1-like peptide ATP substrate binding site wherein the binding site is defined by the atoms found in the three-dimensional atomic coordinates of the Wee1 peptide as set forth in any one of Tables 1-5, or in a related set of atomic coordinates having a root mean square deviation of not more than preferably about 1.25 Å away from the binding site C alpha atoms of an ATP substrate binding site as defined above.

Additionally, the invention also provides a machine-readable medium having stored thereon data comprising the three-dimensional atomic coordinates as set forth in any one of Tables 1-5, or a related set of atomic coordinates having a root mean square deviation of not more than preferably about 1.25 Å away from the core C alpha atoms of the three-dimensional atomic coordinates as set forth in any one of Tables 1-5.

The invention further provides methods having utility in the iterative drug design process. The process identifies potential inhibitors of the Wee1 kinase by structure based drug design, including the use of de novo design of novel drug candidate molecules that bind to and may inhibit Wee1 activity. The three-dimensional atomic coordinates disclosed herein in Tables 1-5 allow for the generation of three-dimensional representations of Wee1 peptide, a structurally related peptide, and the Wee1 peptide ATP substrate binding site or Wee1-like peptide ATP substrate binding site. Thus, the invention provides a method of generating three-dimensional representations of a Wee1 peptide, a structurally related peptide, and/or the Wee1 peptide ATP substrate binding site or Wee1-like peptide ATP substrate binding site defined above, involving applying the three-dimensional atomic coordinates set forth in any one of Tables 1-5, or a related set of atomic coordinates having a root mean square deviation of not more than preferably about 1.25 Å away from the core C alpha atoms of the three-dimensional atomic coordinates as set forth in any one of Tables 1-5, to a computer algorithm to generate a three-dimensional representation (also referred to as an “image”) of a peptide or ATP substrate binding site of the invention.

The three-dimensional atomic coordinates, three-dimensional representations and Wee1 peptide structural information derived therefrom, including the definition of the ATP substrate binding site defined above, may be used, for example, to modify, design, screen and identify, and evaluate chemical entities that have the potential to associate with Wee1 peptide, or a structurally related peptide, and thus have the potential to inhibit activity. For example, structure-based or rational drug design allows the generation of molecules via, for example, the use of computer programs which build and link fragments or atoms into a site based upon steric and electrostatic complementarity to the peptide, without reference to substrate analog structures.

In particular, the invention provides methods for modifying or designing a chemical entity having the potential to associate with a peptide of the invention. The methods include generating a three-dimensional computer representation of a peptide of the invention or an ATP substrate binding site of the invention and generating a chemical entity that spatially conforms to the three-dimensional representation of the peptide or the ATP substrate binding site. The chemical entity may be generated by a method comprising (i) assembling molecular fragments into the chemical entity; (ii) de novo design of the chemical entity or a fragment thereof; (iii) selecting the chemical entity from a small molecule database; or (iv) modifying a known inhibitor, or portion thereof, which possesses the ability to associate with either Wee1 or the structurally related peptide.

The invention also provides methods for screening and identifying a potential inhibitor of the activity of a peptide of the invention. The methods include generating a three-dimensional computer representation of a peptide of the invention or an ATP substrate binding site of the invention; applying an iterative process whereby a chemical entity is applied to the three-dimensional representation to determine whether the chemical entity associates with the peptide or ATP substrate binding site; and evaluating the effect(s) of the chemical entity on peptide activity to determine whether the chemical entity functions as an activity inhibitor.

Methods for evaluating the potential of a chemical entity to associate with a peptide according to the invention are also provided. The methods include generating a three-dimensional representation of a peptide of the invention or an ATP substrate binding site of the invention; applying a three-dimensional representation of a chemical entity to the three-dimensional representation; and quantifying the association between the chemical entity and the binding site.

Additionally, the invention provides methods of utilizing molecular replacement to obtain structural information about a molecule or a molecular complex of unknown structure comprising crystallizing said molecule or molecular complex; generating an X-ray diffraction pattern from said crystallized molecule or molecular complex; and applying at least a portion of the three-dimensional atomic coordinates set forth in any one of Tables 1-5, or a set of atomic coordinates with a root mean square deviation of preferably not more than about 1.25 Å from the core C alpha atoms of the three-dimensional atomic coordinates as set forth in any one of Tables 1-5, to the X-ray diffraction pattern to generate a three-dimensional electron density map of at least a portion of the molecule or molecular complex whose structure is unknown.

### BRIEF DESCRIPTION OF THE DRAWINGS

The patent or patent application file contains at least one drawing executed in color. Copies of this patent or patent application publication with color drawings will be provided by the Office upon request and payment of the necessary fee.

FIG. 1. FIG. 1 is a ribbon representation of Wee1₂₉₁₋₅₇₅ in complex with a 9-Hydroxy-4-phenyl-6H-pyrrolo[3,4-c]carbazole-1,3-dione inhibitor and Magnesium (Mg). The figure shows the canonical view of three-dimensional structure of the modified Wee1 peptide in a complex with the inhibitor. The alpha helical regions of the peptide are colored purple, the beta sheet regions are colored yellow, the Mg atom is colored green and the 9-Hydroxy-4-phenyl-6H-pyrrolo[3,4-c]carbazole-1,3-dione inhibitor is colored orange. Note that the inhibitor molecule is bound at the active site cleft (the ATP substrate binding site) between the N- and C-terminal lobes as found in other kinase structures.

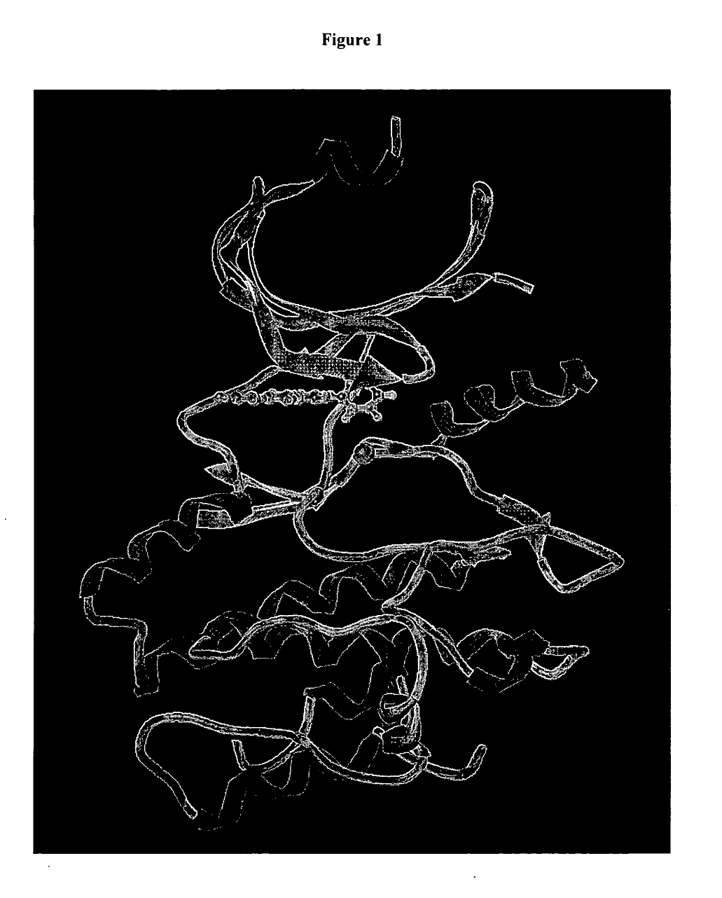

FIG. 2. FIG. 2 is a ribbon representation of Wee1₂₉₁₋₅₇₅ structure in complex with 9-Hydroxy-4-phenyl-6H-pyrrolo[3,4-c]carbazole-1,3-dione inhibitor and Mg, shown rotated about the vertical axis. This Figure uses the same color scheme as in FIG. 1 but is rotated 90° about the vertical axis and provides an alternate view of the location relative to FIG. 1 of the inhibitor with respect to the Mg and the terminal two lobes.

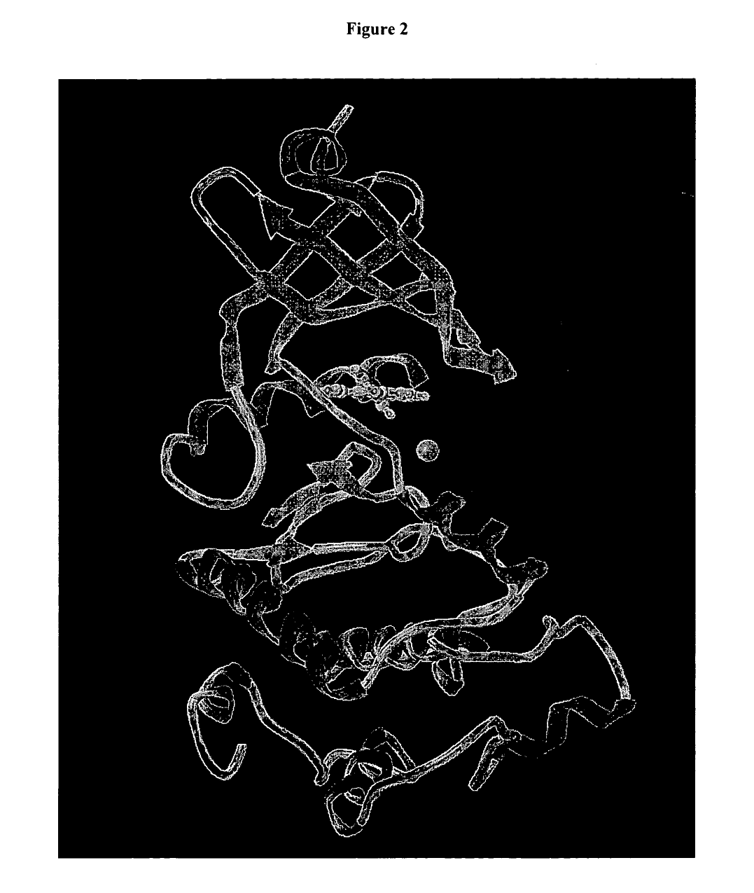

FIG. 3. FIG. 3 is an overlay of the three-dimensional structure of Wee1₂₉₁₋₅₇₅ with five different inhibitors bound to the Wee1 peptide. A surface color-coded by electrostatic potential has been added, calculated from the crystal structure of the Wee1 peptide: 9-Hydroxy-4-phenyl-6H-pyrrolo[3,4-c]carbazole-1,3-dione complex. Dark violet are areas of negative charge, cyan to aqua are slightly negative to neutral, and orange to red areas are positively charged. The Mg atom is colored green, several of the bound waters from the Wee1 peptide: 9-Hydroxy-4-phenyl-6H-pyrrolo[3,4-c]carbazole-1,3-dione complex are in yellow, and the inhibitors are color coded as follows: 9-Hydroxy-4-phenyl-6H-pyrrolo[3,4-c]carbazole-1,3-dione is colored orange; 4-(2-Chloro-phenyl)-8-(dimethylamino-propoxy)-9-hydroxy-6-methyl-6H-pyrrolo[3,4-c]carbazole-1,3-dione is colored aqua; 3-(9-Hydroxy-1,3-dioxo-4-phenyl-2,3-dihydro-1H-pyrrolo[3,4-c]carbazol-6-yl)-propionic acid is colored red; 9-Hydroxy-6-(3-hydroxy-propyl)-4-(2-methoxy-phenyl)-6H-pyrrolo[3,4-c]carbazole-1,3-dione is colored white; and 8-(3-Amino-pyrrolidine-1-carbonyl)-4-(2-chloro-phenyl)-6-methyl-6H-pyrrolo[3,4-c]carbazole-1,3-dione is colored blue. Note that the phenylcarbazole ring systems are essentially co-located, while the side-chain groups may adopt different conformations within the cleft.

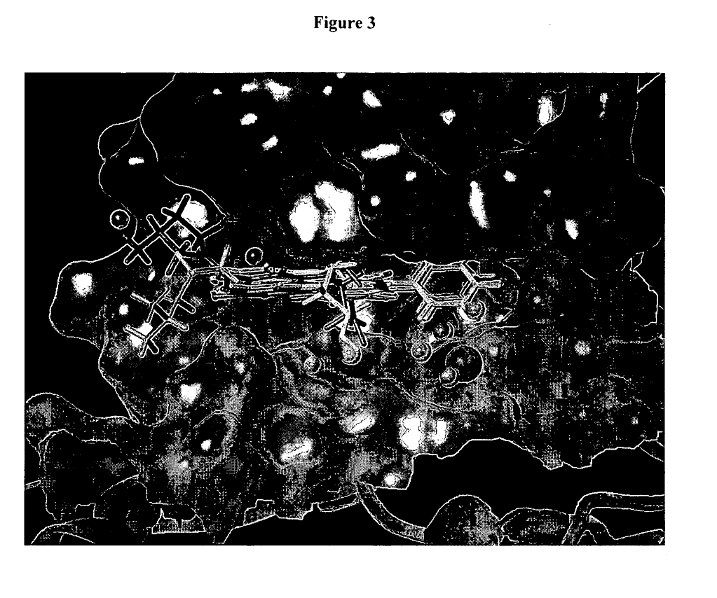

FIG. 4. FIG. 4 is a 90 degree rotated version of FIG. 3, showing the extent of the cleft. Color coding is the same as for FIG. 3.

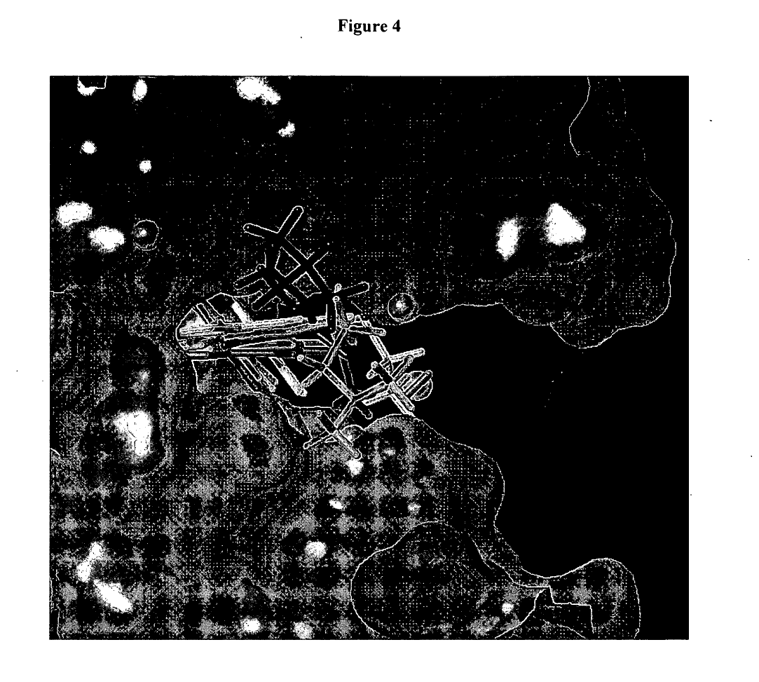

FIG. 5. FIG. 5 depicts the interacting residues surrounding 9-Hydroxy-4-phenyl-6H-pyrrolo[3,4-c]carbazole-1,3-dione as it is bound in the Wee1 ATP substrate binding site. The following residues are found within 4 angstroms of the 9-Hydroxy-4-phenyl-6H-pyrrolo[3,4-c]carbazole-1,3-dione inhibitor located in the ATP substrate binding site: I305, G306, V313, A326, K328, E346, V360, I374, N376, E377, Y378, C379, N380, G382, F433, D463. Within 5 angstroms are the above residues, plus G381, S383, D386, N431, G462, L464, G465, and a Mg atom present in the complex (not shown in FIG. 5). The side chain atoms of E377 and C379 have been removed for clarity. Only polar hydrogens are shown. Hydrogen bonds formed between inhibitor and the Wee1 peptide and bound water molecules within 4 angstroms of the inhibitor are shown (yellow dashed lines). Hydrogen bonds are formed between the inhibitor and the backbone NH and CO of C379, the backbone CO of E377, and the side chain amine of N376. A pi stacking interaction is apparent between the inhibitor and F433, and hydrophobic interactions are present between inhibitor the side chains of I305, V313, A326, and to some extent, Y378. A pocket of sufficient size to accommodate a 4-phenyl group, or group of similar size, within the inhibitors is formed by V313, K328, E346, D463, N376, I374, and to some extent, the bound Magnesium atom.

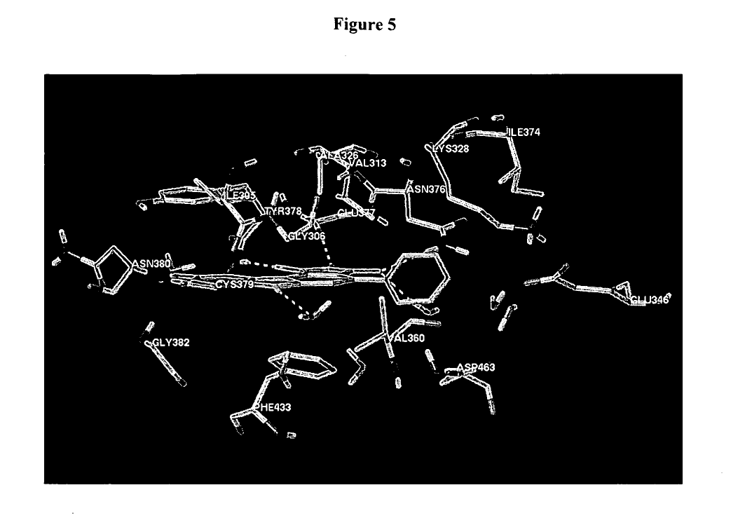

### Sequence Listing

SEQ ID NO: 1 full length Wee1 nucleic acid sequence

SEQ ID NO: 2 fall length Wee1 peptide sequence encoded by SEQ ID NO: 1

SEQ ID NO: 3 nucleotide sequence for human Wee1 kinase domain forward primer

SEQ ID NO: 4 amino acid sequence corresponding to SEQ ID NO: 3

SEQ ID NO: 5 nucleotide sequence for human Wee1 kinase domain reverse primer

SEQ ID NO: 6 amino acid sequence amplified by the primer of SEQ ID NO: 5

SEQ ID NO: 7 Wee1₂₉₁₋₅₇₅ peptide & primer amino acid sequence with stop codon

SEQ ID NO: 8 Wee1₂₉₁₋₅₇₅ peptide with GAMG at NH₂-terminal end and stop codon

### DETAILED DESCRIPTION OF PREFERRED EMBODIMENTS

The present invention provides modified Wee1 peptides, crystalline structures comprising a binary complex of a modified Wee1 peptide, or a peptide that is structurally related to Wee1 peptide, and an inhibitor, and the resulting three-dimensional atomic coordinates. The resulting three-dimensional crystallographic structural information for the Wee1 complexes, including an ATP substrate binding site of the Wee1 peptide or a peptide that is structurally related to Wee1 peptide, may be used in, for example, the modification, design, screening and identification, and evaluation of chemical entities that have the potential to associate with Wee1 peptide and thus may inhibit Wee1 activity. The structural information may also be used to modify, design, screen and identify, and evaluate chemical entities that have the potential to associate with a peptide that is structurally related to Wee1 peptide. The chemical entities (also referred to as “candidate compounds”) have the potential to be therapeutic agents for the treatment of various diseases associated with Wee1 peptide. For example, inhibition of the Wee1 enzyme has been found to impair proliferation and affect a number of potential disease states that involve diverse cellular processes including apoptosis, differentiation, angiogenisis and inflammation. As a result, inhibitors are expected to be effective in the treatment of a wide variety of diseases, including, but not limited to cancer, psoriasis, arthritis, septic shock, viral infections and cardiovascular disease.

Definitions

As used herein, the terms “comprising” and “including” are used in the conventional, non-limiting sense.

As used herein, “ligand” means a small molecule that binds to or associates specifically with an enzyme and includes, for example, an inhibitor.

As used herein, “cofactor” or “substrate” mean an inorganic molecule, an organic molecule or a coenzyme that is required for enzymatic activity. For example, ATP (adenosine triphosphate) is a substrate used by a kinase enzyme to transfer a phosphate group, i.e. phosphorylate its peptide substrate.

As used herein, the phrase “root mean square (RMS) deviation” denotes the structural relationship between two or more species of proteins or peptides. It means that the difference in the root mean square of the distance of the three-dimensional structure of one peptide from C-alpha (C_{α}) atoms or backbone trace of the Wee1 peptide in units of Angstroms (Å) unless indicated otherwise. It may be determined by superimposing one of the three-dimensional structures of the species on another, which may be solved by, for example, X-ray crystallography or by NMR and measuring the difference in the root mean square of the distance from C_{α} atoms or backbone trace of the Wee1 peptide to the other peptide in units of Angstroms (Å). The superimposing of three-dimensional structures on one another may be performed using a molecular modeling program, for example CNX™ (Accelrys), XtalView™ (Duncan McRee, Scripps Research Institute) or O™ (Morten Kjeldgaard, Aarhus Univ., Denmark). The closer the relationship between the three-dimensional structures, the smaller will be the value of the RMS deviation. For example, the three-dimensional relationship between the three-dimensional atomic coordinates of the C-alpha atoms of two inhibitor protein co-complex structures is typically between 0.0-0.5 Å RMS deviation.

Therefore, one embodiment of this invention is the three-dimensional structures of the modified Wee1 peptides in binary complexes with an inhibitor as found in Table 1, Table 2, Table 3, Table 4, and Table 5. An additional embodiment is a “structurally related” peptide, crystals of the structurally related peptide and the three-dimensional structures thereof. As used herein, a “structurally related” protein or peptide refers to a protein or peptide that is defined by the three-dimensional atomic coordinates of the Wee1 peptide as set forth in Table 1, Table 2, Table 3, Table 4, or Table 5, or by a related set of atomic coordinates having a root mean square deviation of from not more than about 1.5 Å to not more than about 0.50 Å from the core C alpha atoms of the three-dimensional atomic coordinates as set forth in Table 1, Table 2, Table 3, Table 4, or Table 5. Preferably the root mean square deviation is not more than about 0.50 Å, more preferably not more than about 0.75 Å, even more preferably not more than about 1.00 Å, and most preferably not more than about 1.25 Å.

Similarly, as used herein, “related set of atomic coordinates” refers to a set of atomic coordinates having a root mean square deviation in the range of from not more than about 1.5 Å to not more than about 0.50 Å away from the core C alpha atoms of the three-dimensional atomic coordinates as set forth in Table 1, Table 2, Table 3, Table 4, or Table 5. Preferably the root mean square deviation is not more than about 0.50 Å, more preferably not more than about 0.75 Å, even more preferably not more than about 1.00 Å, and most preferably not more than about 1.25 Å.

As used herein, “chemical entity” refers to a chemical compound, a complex of at least two chemical compounds, or a fragment of such a compound or complex. Such entities can be, for example, potential drug candidates and can be evaluated for their ability to inhibit the activity of Wee1, or a structurally related peptide.

As used herein, the term “inhibitor” or “inhibit” (or variations thereof) refers to a ligand, such as a compound or substance that lowers, reduces, decreases, prevents, diminishes, stops or negatively interferes with Wee1's activity, or such actions. Often the terms “inhibitor” and “antagonist” can be used interchangeably. Inhibition is typically expressed as a percentage of the enzyme activity in the presence of the inhibitor over the enzyme activity without the inhibitor. Or it may be expressed in terms of IC₅₀, the inhibitor concentration at which 50% of the original enzyme activity is observed.

Wee1

As used herein, the abbreviation “Wee1” refers to the polynucleotide encoding the Wee1 (also referred to as “Wee1-Hu”) kinase, or the peptide per se. The nucleic acid sequence of the polynucleotide encoding the full-length protein of Wee1 was published by Igarashi et al., submitted to GenBank and assigned accession number X62048. The nucleic acid sequence described therein is provided herein, shown in SEQ ID NO: 1. The corresponding amino acid sequence encoded by the nucleic acid sequence of SEQ ID NO: 1 is provided herein, shown in SEQ ID NO: 2. This peptide sequence was submitted to GenBank by Igarashi and assigned Accession number CAA43979.

As used herein, “Wee1₂₉₁₋₅₇₅” refers to a Wee1 peptide having amino acid residues 291-575 of SEQ ID NO: 2 or a conservatively substituted variant thereof.

As used herein, “Wee1₂₉₁₋₆₀₆” refers to a Wee1 peptide having amino acid residues 291-606 of SEQ ID NO: 2 or a conservatively substituted variant thereof.

Wee1 Peptide ATP Substrate Binding Site

As used herein, “binding site,” also referred to as, for example, “binding pocket,” “binding domain,” “substrate binding site,” “catalytic domain,” or “inhibitor-binding domain,” “inhibitor-binding site,” refers to a region or regions of a molecule or molecular complex, that, as a result of its surface features, including, but not limited to, volume (both internally in cavities or in total), solvent accessibility, and surface charge and hydrophobicity, can associate with another chemical entity or compound. Such regions are of utility in fields such as drug discovery.

It has been discovered that the Wee1 peptide comprises an ATP substrate binding site that is defined by the three-dimensional atomic coordinates of the following amino acid residues within about 4 Å of an inhibitor located in the ATP substrate binding site: I305, G306, V313, A326, K328, E346, V360, I374, N376, E377, Y378, C379, N380, G382, F433, and D463 of SEQ ID NO: 2, or a conservatively substituted variant thereof; and by the three-dimensional atomic coordinates of the following amino acid residues within about 5 Å of an inhibitor located in the ATP substrate binding site: I305, G306, V313, A326, K328, E346, V360, I374, N376, E377, Y378, C379, N380, G381, G382, S383, D386, N431, F433, G462, D463, L464, G465 of SEQ ID NO: 2, or a conservatively substituted variant thereof. The ATP substrate binding site is also defined by the following amino acid residues I305, V313, A326, N376, E377, Y378, C379, G382, and F433 of SEQ ID NO: 2, or a conservatively substituted variant thereof. Additionally, the ATP substrate binding site is defined by V313, K328, E346, D463, N376, and I374 of SEQ ID NO: 2, or a conservatively substituted variant thereof. Optionally, the binding site may additionally be defined by tightly bound water molecules, and/or a bound Magnesium atom and its associated waters.

Accordingly, the invention includes isolated and purified polypeptides consisting of an ATP substrate binding site defined by:

(1) the three-dimensional atomic coordinates of the following amino acid residues within about 4 Å of an inhibitor located in the ATP substrate binding site: I305, G306, V313, A326, K328, E346, V360, I374, N376, E377, Y378, C379, N380, G382, F433, D463 of SEQ ID NO: 2, or a conservatively substituted variant thereof;

(2) the three-dimensional atomic coordinates of the following amino acid residues within about 5 Å of an inhibitor located in the ATP substrate binding site: I305, G306, V313, A326, K328, E346, V360, I374, N376, E377, Y378, C379, N380, G381, G382, S383, D386, N431, F433, G462, D463, L464, G465 of SEQ ID NO: 2, or a conservatively substituted variant thereof.

(3) the following amino acid residues: I305, V313, A326, N376, E377, Y378, C379, G382, and F433 of SEQ ID NO: 2, or a conservatively substituted variant thereof; or

(4) the following amino acid residues: V313, K328, E346, D463, N376, and I374 of SEQ ID NO: 2, or a conservatively substituted variant thereof (optionally, the binding site may additionally be defined by tightly bound water molecules and/or a bound Magnesium atom and its associated waters).

As used herein, a “Wee1-like” peptide ATP substrate binding site refers to an ATP substrate binding site defined by the atoms found in the three-dimensional atomic coordinates of the Wee1 peptide as set forth in Table 1, Table 2, Table 3, Table 4, or Table 5, or defined by atomic coordinates having a root mean square deviation of from not more than about 1.5 Å to not more than about 0.50 Å, preferably of not more than about 1.25 Å, away from the ATP substrate binding site C alpha atoms of any one of the Wee1 ATP substrate binding site definitions provided herein, or a conservatively substituted variant thereof.

As used herein, the term “activity” refers to all activities, i.e., the function of Wee1 in the phosphorylation of its substrate Cdc2/cyclinB, as well as to the enzyme's potency. Often the terms “activity” and “function” can be used interchangeably.

As used herein, the term “associate” refers to the process in which at least two molecules reversibly interact with each other, for example, by binding with each other. This may also refer to the process in which the conformation of the protein changes in response to the presence of an inhibitor to better accommodate the steric and electrostatic effects of the inhibitor. Associations between Wee1 peptide, or a structurally related peptide, and an inhibitor may occur with all or a part of a Wee1 or Wee1-like peptide ATP substrate binding site. The association(s) may be non-covalent, e.g., wherein the juxtaposition is energetically favored by hydrogen bonding, van der Waals interactions or electrostatic interactions, or the association(s) may be covalent.

As used herein, the terms “model” and “modeling” mean the procedure of evaluating (also referred to as “assessing”) the affinity of the interaction between a Wee1 or Wee1-like peptide ATP substrate binding site and a chemical entity (also referred to as a “candidate compound”) based on steric constraints and surface/solvent electrostatic effects.

Wee1 Peptide

The present invention provides isolated peptide and protein molecules that consist of, consist essentially of or are comprised of the amino acid sequences of the peptides encoded by the nucleic acid sequences disclosed in the SEQ ID NO: 1 or fragments thereof, as well as obvious variants of these peptides that are within the art to make and use. Some of these variants are described in detail below.

In one embodiment, the invention provides an isolated, substantially pure polypeptide comprising novel modified Wee1 peptides, constructed, for example, so as to omit a significant portion of the NH₂-terminal and COOH-terminal tails of the full length Wee1 peptide, or a conservatively substituted variant thereof (“modified Wee1 peptides”). The modified Wee1 peptides of the invention preferably retain the globular core of the corresponding full-length Wee1 peptide, such that Wee1 can bind to ATP, its natural substrate. Preferably, the modified Wee1 peptides lack (1) all or a significant portion of the NH₂-terminus, preferably up to about 290 amino acids; and (2) all or a significant portion of the COOH-terminus, preferably up to about 70 amino acids. More preferably, the modified Wee1 peptide lack 290 amino acid residues from the NH₂-terminal region of the full length Wee1 peptide and 70 amino acid residues from the COOH-terminal region of the full length Wee1 peptide. Thus, the preferred modified Wee1 peptide has an amino acid sequence from amino acid 291 to amino acid 575 of SEQ ID NO: 2 or a conservatively substituted variant thereof.

Additionally, the modified Wee1 peptides may have a histidine oligomer tag, for example a histidine hexamer tag (“His-tag”), at their NH₂-terminal end and/or a primer at the NH₂-terminal end, such as, for example, a primer having the amino acid sequence GAMG.

The modified Wee1 peptides of the present invention can be derived from any eukaryotic source, preferably from a vertebrate Wee1, more preferably from a mammalian Wee1, and most preferably from human Wee1.

As used herein, a protein or peptide is said to be “isolated” or “purified” when it is substantially free of cellular material or free of chemical precursors or other chemicals. The proteins or peptides of the present invention can be purified to homogeneity or other degrees of purity. The level of purification will be based on the intended use. The critical feature is that the preparation allows for the desired function of the protein or peptide, even if in the presence of considerable amounts of other components.

In some uses, “substantially free of cellular material” includes preparations of the protein or peptide having less than about 30% (by dry weight) other proteins (i.e., contaminating protein), preferably less than about 20% other proteins, more preferably less than about 10% other proteins, or even more preferably less than about 5% other proteins. When the protein or peptide is recombinantly produced, it can also be substantially free of culture medium, i.e., culture medium represents less than about 20% of the volume of the protein preparation.

The language “substantially free of chemical precursors or other chemicals” includes preparations of the protein in which it is separated from chemical precursors or other chemicals that are involved in its synthesis. In one embodiment, the language “substantially free of chemical precursors or other chemicals” includes preparations of the protein having less than about 30% (by dry weight) chemical precursors or other chemicals, preferably less than about 20% chemical precursors or other chemicals, more preferably less than about 10% chemical precursors or other chemicals, or most preferably less than about 5% chemical precursors or other chemicals.

The isolated protein described herein can be purified from cells that naturally express Wee1 or purified from cells that have been altered to express Wee1 (recombinant expression). For example, a nucleic acid molecule encoding the protein is cloned into an expression vector, the expression vector is introduced into a host cell and the protein is then expressed in the host cell. The protein can then be isolated from the cells by an appropriate purification scheme using standard protein purification techniques. Many of these techniques are described in detail below.

“Polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. The terms “peptide”, “polypeptide” and “protein” are used interchangeably herein. Polypeptides may contain amino acids other than the 20 naturally occurring amino acids. Further, many amino acids, including the terminal amino acids, may be modified by natural processes, such as processing and other post-translational modifications, or by chemical modification techniques well known in the art. Common modifications that occur naturally in polypeptides are described in basic texts, detailed monographs, and the research literature, and they are well known to those of skill in the art.

Accordingly, the polypeptides also encompass derivatives or analogs in which a substituted amino acid residue is not one encoded by the genetic code; in which a substituent group is included, in which the mature polypeptide is fused with another compound, such as a compound to increase the half-life of the polypeptide (for example, polyethylene glycol), or in which the additional amino acids are fused to the mature polypeptide, such as a leader or secretary sequence or a sequence for purification of the mature polypeptide or a pro-protein sequence.

The present invention further provides for fragments of the Wee1 peptide, in addition to proteins and peptides that comprises and consist of such fragments. As used herein, a fragment comprises at least 8 or more contiguous amino acid residues from the protein. Such fragments can be chosen based on the ability to retain one or more of the biological activities of the kinase or could be chosen for the ability to perform a function, e.g. act as an immunogen. Particularly important fragments are biologically active fragments, peptides that are, for example about 8 or more amino acids in length. Such fragments will typically comprise a domain or motif of the kinase, e.g., active site. Further, possible fragments include, but are not limited to, domain or motif containing fragments, soluble peptide fragments, and fragments containing immunogenic structures. Predicted domains and functional sites are readily identifiable by computer programs well known and readily available to those of skill in the art (e.g., by PROSITE analysis).

Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, disulfide bond formation, demethylation, formation of covalent crosslinks, formation of cystine, formation of pyroglutamate, formylation, gamma carboxylation, glycosylation, GPI anchor formation, hydroxylation, iodination, methylation, myristoylation, oxidation, proteolytic processing, phosphorylation, phenylation, racemization, selenoylation, sulfation, transfer-RNA mediated addition of amino acids to proteins such as arginylation, and ubiquitination.

Such modifications are well known to those of skill in the art and have been described in great detail in the scientific literature. Several particularly common modifications, glycosylation, lipid attachment, sulfation, gamma-carboxylation of glutamic acid residues, hydroxylation and ADP-ribosylation, for instance, are described in most basic texts. For example, see Proteins—Structure and Molecular Properties, 2nd Ed., T. E. Creighton, W. H. Freeman and Company, New York (1993). Many detailed reviews are available on this subject, such as by Wold, F., Posttranslational Covalent Modification of Proteins, B. C. Johnson, Ed., Academic Press, New York 1-12 (1983); Seifter et al. (Meth. Enzymol. 182: 626-646 (1990)) and Rattan et al. (Ann. N.Y. Acad. Sci. 663: 48-62 (1992)).

The peptides of the present invention can be attached to heterologous sequences to form chimeric or fusion proteins. Such chimeric and fusion proteins comprise a peptide operatively linked to a heterologous protein having an amino acid sequence not substantially homologous to the kinase peptide. “Operatively linked” indicates that the peptide and the heterologous protein are fused in-frame. The heterologous protein can be fused to the NH₂-terminus or COOH-terminus of the kinase peptide. The two peptides linked in a fusion peptide are typically derived from two independent sources, and therefore a fusion peptide comprises two linked peptides not normally found linked in nature. The two peptides may be from the same or different genome.

In some uses, the fusion protein does not affect the activity of the peptide per se. For example, the fusion protein can include, but is not limited to, enzymatic fusion proteins, for example beta-galactosidase fusions, yeast two-hybrid GAL fusions, poly-His fusions, MYC-tagged, HI-tagged and Ig fusions. Such fusion proteins, particularly poly-His fusions, can facilitate the purification of recombinant kinase peptide. In certain host cells (e.g., mammalian host cells), expression and/or secretion of a protein can be increased by using a heterologous signal sequence.

A chimeric or fusion protein can be produced by standard recombinant DNA techniques. For example, DNA fragments coding for the different protein sequences are ligated together in-frame in accordance with conventional techniques. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed and re-amplified to generate a chimeric gene sequence (see Ausubel et al., Current Protocols in Molecular Biology, 1992). Moreover, many expression vectors are commercially available that already encode a fusion moiety (e.g., a GST protein). A modified Wee1 peptide-encoding nucleic acid can be cloned into such an expression vector such that the fusion moiety is linked in-frame to the peptide.

Nucleic Acids and Polynucleotides Encoding Modified Wee1

The present invention also provides isolated nucleic acid molecules that encode the functional, active kinases of the present invention. Such nucleic acid molecules will consist of, consist essentially of, or comprise a nucleotide sequence that encodes one of the kinase peptides of the present invention, an allelic variant thereof, or an ortholog or paralog thereof. In a particular embodiment, the invention also provides isolated, purified polynucleotides that encode novel modified Wee1 peptides, such as the modified Wee1 peptides of the invention.

The polynucleotide may be natural or recombinant. In the preferred embodiment, the nucleic acid sequence encodes a modified Wee1 peptide having an amino acid sequence from amino acid 291 to amino acid 575 of SEQ ID NO: 2 or a conservatively substituted variant thereof. Such polynucleotides may be, for example, one having the sequence set forth in SEQ ID NO: 1 with deletion of the nucleotides encoding residues 1 to 290 of SEQ ID NO:2 and the 5′-noncoding region and with deletion of the nucleotides encoding residues 576 to 646 of SEQ ID NO: 2 and the 3′-noncoding region.

As used herein, an “isolated” nucleic acid molecule is one that is separated from other nucleic acid present in the natural source of the nucleic acid. Preferably, an “isolated” nucleic acid is free of sequences which naturally flank the nucleic acid (i.e., sequences located at the 5′ and 3′ ends of the nucleic acid) in the genomic DNA or cDNA of the organism from which the nucleic acid is derived. However, there can be some flanking nucleotide sequences, for example up to about 5 KB, particularly contiguous peptide encoding sequences and peptide encoding sequences within the same gene but separated by introns in the genomic sequence. The important point is that the nucleic acid is isolated from remote and unimportant flanking sequences such that it can be subjected to the specific manipulations described herein such as recombinant expression, preparation of probes and primers, and other uses specific to the nucleic acid sequences.

Moreover, an “isolated” nucleic acid molecule, such as a cDNA molecule, can be substantially free of other cellular material, or culture medium when produced by recombinant techniques, or chemical precursors or other chemicals when chemically synthesized. However, the nucleic acid molecule can be fused to other coding or regulatory sequences and still be considered isolated.

For example, recombinant DNA molecules contained in a vector are considered isolated. Further examples of isolated DNA molecules include recombinant DNA molecules maintained in heterologous host cells or purified (partially or substantially) DNA molecules in solution. Isolated RNA molecules include in vivo or in vitro RNA transcripts of the isolated DNA molecules of the present invention. Isolated nucleic acid molecules according to the present invention further include such molecules produced synthetically.

The preferred classes of nucleic acid molecules that are comprised of the nucleotide sequences of the present are the full-length cDNA molecules and genes and genomic clones since some of the nucleic acid molecules provided in SEQ ID NO: 1 are fragments of the complete gene that exists in nature. A brief description of how various types of these nucleic acid molecules can be readily made/isolated is provided herein.

Full-length genes may be cloned from known sequences using any one of a number of methods known in the art. For example, a method which employs XL-PCR (Perkin-Elmer, Foster City, Calif.) to amplify long pieces of DNA may be used. Other methods for obtaining full-length sequences are known in the art.

The isolated nucleic acid molecules can encode the active protein plus additional amino or carboxyl-terminal amino acids, or amino acids interior to the mature peptide (when the mature form has more than one peptide chain, for instance). Such sequences may play a role in processing of a protein from precursor to an active form, facilitate protein trafficking, prolong or shorten protein half-life or facilitate manipulation of a protein for assay or production, among other things. As generally is the case in situ, the additional amino acids may be processed away from the mature active protein by cellular enzymes.

As mentioned above, the isolated nucleic acid molecules include, but are not limited to, the sequence encoding the active kinase alone or in combination with coding sequences, such as a leader or secretary sequence (e.g., a pre-pro or pro-protein sequence), the sequence encoding the active kinase, with or without the additional coding sequences, plus additional non-coding sequences, for example introns and non-coding 5′ and 3′ sequences such as transcribed but non-translated sequences that play a role in transcription, mRNA processing (including splicing and polyadenylation signals), ribosome binding and stability of mRNA. In addition, the nucleic acid molecule may be fused to a marker sequence encoding, for example, a peptide that facilitates purification.

Isolated nucleic acid molecules can be in the form of RNA, such as mRNA, or in the form of DNA, including cDNA and genomic DNA, obtained by cloning or produced by chemical synthetic techniques or by a combination thereof. The nucleic acid, especially DNA, can be double-stranded or single-stranded. Single-stranded nucleic acid can be the coding strand (sense strand) or the non-coding strand (anti-sense strand).

The invention further provides nucleic acid molecules that encode fragments of the peptides of the present invention and that encode obvious variants of the kinase proteins of the present invention that are described above. Such nucleic acid molecules may be naturally occurring, such as allelic variants (same locus), paralogs (different locus), and orthologs (different organism), or may be constructed by recombinant DNA methods or by chemical synthesis. Such non-naturally occurring variants may be made by mutagenesis techniques, including those applied to nucleic acid molecules, cells, or organisms. Accordingly, as discussed below, the variants can contain nucleotide substitutions, deletions, inversions and insertions. Variation can occur in either or both the coding and non-coding regions. The variations can produce both conservative and non-conservative amino acid substitutions.

A fragment comprises a contiguous nucleotide sequence greater than 12 or more nucleotides. Further, a fragment could be at least 30, 40, 50, 100, 250 or 500 nucleotides in length. The length of the fragment will be based on its intended use. For example, the fragment can encode epitope bearing regions of the peptide, or can be useful as DNA probes and primers. Such fragments can be isolated using the known nucleotide sequence to synthesize an oligonucleotide probe. A labeled probe can then be used to screen a cDNA library, genomic DNA library, or mRNA to isolate nucleic acid corresponding to the coding region. Further, primers can be used in PCR reactions to clone specific regions of gene.

A probe/primer typically comprises substantially a purified oligonucleotide or oligonucleotide pair. The oligonucleotide typically comprises a region of nucleotide sequence that hybridizes under stringent conditions to at least about 12, 20, 25, 40, 50 or more consecutive nucleotides.

Orthologs, homologs, and allelic variants can be identified using methods known in the art. As described above, these variants comprise a nucleotide sequence encoding a peptide that is typically 60%, preferably 65%, more preferably 70%, or even more preferably 75% or more homologous to the nucleotide sequence provided in SEQ ID NO: 1 or a fragment of this sequence. In one preferred embodiment, the variants comprise a nucleotide sequence encoding a peptide that is at least 80%, preferably 85%, more preferably 90%, even more preferably 95% or more homologous to the nucleotide sequence provided in SEQ ID NO: 1 or a fragment of this sequence. Such nucleic acid molecules can readily be identified as being able to hybridize under moderate to stringent conditions, to the nucleotide sequence shown in SEQ ID NO: 1 or a fragment of the sequence.

As used herein, the phrase “hybridize(s) under stringent conditions” is intended to describe conditions for hybridization and washing under which nucleotide sequences encoding a peptide at least 50%, preferably at least 55%, homologous to each other typically remain hybridized to each other. The conditions can be such that sequences at least 65%, preferably at least 70%, or more preferably at least 75% homologous to each other typically remains hybridized to each other. Standard hybridization conditions from moderate to highly stringent conditions are known to those skilled in the art (See e.g., Current Protocols in Molecular Biology, John Wiley & Sons, N.Y. (1989), 6.3.1-6.3.6). Moderate hybridization conditions are defined as equivalent to hybridization in 2×sodium chloride/sodium citrate (SSC) at 30° C., followed by one or more washes in 1×SSC, 0.1% SDS at 50-60° C. Highly stringent conditions are defined as equivalent to hybridization in 6×sodium chloride/sodium citrate (SSC) at 45° C., followed by one or more washes in 0.2×SSC, 0.1% SDS at 50-65° C.

The nucleic acid molecules of the present invention are useful for probes, primers, chemical intermediates, and in biological assays. The nucleic acid molecules are useful as a hybridization probe for cDNA and genomic DNA to isolate full-length cDNA and genomic clones encoding the peptide described herein and to isolate cDNA and genomic clones that correspond to variants (alleles, orthologs, etc.) producing the same or related peptides described herein.

The probe can correspond to any sequence along the entire length of the nucleic acid molecules provided in the SEQ ID NO: 1. Accordingly, it could be derived from 5′ noncoding regions, the coding region, and 3′ noncoding regions. However, fragments are not to be construed as those which may encompass fragments disclosed prior to the present invention.

The nucleic acid molecules are also useful as primers for PCR to amplify any given region of a nucleic acid molecule and are useful to synthesize antisense molecules of desired length and sequence.

The nucleic acid molecules are also usefuil for constructing recombinant vectors. Such vectors include expression vectors that express a portion of, or all of, the peptide sequences. Vectors also include insertion vectors, used to integrate into another nucleic acid molecule sequence, such as into the cellular genome, to alter in situ expression of a gene and/or gene product. For example, an endogenous coding sequence can be replaced via homologous recombination with all or part of the coding region containing one or more specifically introduced mutations. The nucleic acid molecules are also useful for expressing antigenic portions of the proteins; useful as probes for determining the chromosomal positions of the nucleic acid molecules by means of in situ hybridization methods; useful for designing ribozymes corresponding to all, or a part, of the mRNA produced from the nucleic acid molecules described herein; and useful for constructing host cells expressing a part, or all, of the nucleic acid molecules and peptides. The nucleic acid molecules are also useful for constructing transgenic animals expressing all, or a part, of the nucleic acid molecules and peptides; and useful for making vectors that express part, or all, of the peptides. The nucleic acid molecules are further useful as hybridization probes for determining the presence, level, form and distribution of nucleic acid expression. Accordingly, the probes can be used to detect the presence of, or to determine levels of, a specific nucleic acid molecule in cells, tissues, and in organisms. The nucleic acid whose level is determined can be DNA or RNA. Accordingly, probes corresponding to the peptides described herein can be used to assess expression and/or gene copy number in a given cell, tissue, or organism. These uses are relevant for diagnosis of disorders involving an increase or decrease in kinase protein expression relative to normal expression.

In vitro techniques for detection of mRNA include Northern hybridizations and in situ hybridizations. In vitro techniques for detecting DNA includes Southern hybridizations and in situ hybridization.

Vectors and Host Cells for Producing Modified Wee1

The present invention also provides for an expression vector for producing the modified Wee1 peptides described above in a host cell. It further provides a host cell which may be stably transformed and transfected with a polynucleotide encoding the modified Wee1 peptides described above, or a fragment thereof or an analog thereof, in a manner allowing the expression of the modified Wee1 peptides. The present invention also provides expression vectors comprising the nucleic acid of the present invention operatively associated with an expression control sequence. The expression vector may comprise, for example, a nucleic acid encoding a modified Wee1 peptide having an amino acid sequence from amino acid 291 to amino acid 575 of SEQ ID NO: 2, or a conservatively substituted variant thereof.

The present invention further includes a cell transfected or transformed with an expression vector of the present invention. Any of the cells mentioned below may be employed in this method. In one embodiment, the cell is prokaryotic. In another embodiment the cell is an eukaryotic cell, such as an insect cell or a vertebrate cell, which may be, for example, a mammalian cell. Preferably, the cell is an insect cell.

The present invention also includes methods for expressing the modified Wee1 peptide comprising culturing a cell that expresses the modified Wee1 peptide in an appropriate cell culture medium or fermenation medium under conditions that provide for expression of the protein by the cell. Any of the cells mentioned above may be employed in this method. In a particular embodiment, the cell is a yeast cell which has been manipulated to express a modified Wee1 peptide of the present invention. In a preferred embodiment, the cell is a eukaryote, such as, for example, defined insect cell lines that has been manipulated to express a modified Wee1 peptide of the present invention.

The invention also provides vectors containing the nucleic acid molecules described herein. The term “vector” refers to a vehicle, preferably a nucleic acid molecule that can transport the nucleic acid molecules of the invention. When the vector is a nucleic acid molecule, the inventive nucleic acid molecules are covalently linked to the vector nucleic acid. The vector preferably is a plasmid, single or double stranded phage, a single or double stranded RNA or DNA viral vector, or artificial chromosome, such as aBAC, PAC, YAC, or MAC, which are commercially available from, for example, Qiagen (Valencia, Calif.). Various expression vectors known in the art can be used to express polynucleotides encoding the Wee1 peptide or variants thereof.

A vector can be maintained in the host cell as an extrachromosomal element where it replicates and produces additional copies of the inventive nucleic acid molecules. Alternatively, the vector may integrate into the host cell genome and produce additional copies of the inventive nucleic acid molecules when the host cell replicates.

The invention provides vectors for the maintenance (cloning vectors) or vectors for expression (expression vectors) of the inventive nucleic acid molecules. The vectors can function in prokaryotic or eukaryotic cells or in both (shuttle vectors). Expression vectors contain cis-acting regulatory regions that are operably linked in the vector to the inventive nucleic acid molecules such that transcription of the inventive nucleic acid molecules is allowed in a host cell. The inventive nucleic acid molecules can be introduced into the host cell with a separate nucleic acid molecule capable of affecting transcription. Thus, the second nucleic acid molecule may provide a trans-acting factor interacting with the cis-regulatory control region to allow transcription of the inventive nucleic acid molecules from the vector. Alternatively, a trans-acting factor may be supplied by the host cell. Finally, a trans-acting factor can be produced from the vector itself. It is understood, however, that in some embodiments, transcription and/or translation of the inventive nucleic acid molecules can occur in a cell-free system.

The regulatory sequence to which the nucleic acid molecules described herein can be operably linked include promoters for directing mRNA transcription. These include, but are not limited to, the left promoter from bacteriophage λ, the lac, TRP, and TAC promoters from E. coli, the early and late promoters from SV40, the CMV immediate early promoter, the adenovirus early and late promoters, retrovirus long-terminal repeats, p10 and pPOLh promoters from insect viral sources, and tetracycline response elements from inducible mammalian expression systems.

In addition to control regions that promote transcription, expression vectors may also include regions that modulate transcription, such as repressor binding sites and enhancers. Examples include the SV40 enhancer, the cytomegalovirus immediate early enhancer, polyoma enhancer, adenovirus enhancers, retrovirus LTR enhancers, and tetracycline response elements from inducible mammalian expression systems

In addition to containing sites for transcription initiation and control, expression vectors can also contain sequences necessary for transcription termination and, in the transcribed region a ribosome-binding site for translation. Other regulatory control elements for expression include initiation and termination codons as well as polyadenylation signals. The person of ordinary skill in the art would be aware of the numerous regulatory sequences that are useful in expression vectors. Such regulatory sequences are described, for example, in Sambrook et al., (Molecular Cloning: A Laboratory Manual. 2nd. ed., Cold Spring Harbor Laboratory Press, Cold Spring Harbor, N.Y., (1989)).

A variety of expression vectors can be used to express a protein encoded by a nucleic acid molecule. Such vectors include chromosomal, episomal, and virus-derived vectors, for example vectors derived from bacterial plasmids, from bacteriophage, from yeast episomes, from yeast chromosomal elements, including yeast artificial chromosomes, from viruses such as baculoviruses, papovaviruses such as SV40, Vaccinia viruses, adenoviruses, poxviruses, pseudorabies viruses, and retroviruses. Vectors may also be derived from combinations of these sources such as those derived from plasmid and bacteriophage genetic elements, e.g., cosmids and phagemids. Appropriate cloning and expression vectors for prokaryotic and eukaryotic hosts are described in Sambrook et al., Molecular Cloning: A Laboratory Manual. 2nd. ed., Cold Spring Harbor Laboratory Press, Cold Spring Harbor, N.Y., (1989) and in the journal Protein Expression and Purification and references cited therein (Elsevier Science Publishing, USA).

The regulatory sequence may provide constitutive expression in one or more host cells (i.e. tissue specific) or may provide for inducible expression in one or more cell types such as by temperature, nutrient additive, or exogenous factor such as a hormone or other ligand. A variety of vectors providing for constitutive and inducible expression in prokaryotic and eukaryotic hosts are known to those of ordinary skill in the art.

The inventive nucleic acid molecules can be inserted into the vector nucleic acid by well-known methodology. Generally, the DNA sequence that will ultimately be expressed is joined to an expression vector by cleaving the DNA sequence and the expression vector with one or more restriction enzymes and then ligating the fragments together. Procedures for restriction enzyme digestion and ligation are known to those of ordinary skill in the art.

The vector containing the appropriate nucleic acid molecule can be introduced into an appropriate host cell for propagation or expression using well-known techniques. Bacterial cells include, but are not limited to, E. coli, Streptomyces, and Salmonella typhimurium. Eukaryotic cells include, but are not limited to, yeast, insect cells such as Drosophila, Spodoptera frugiperda, animal cells such as COS and CHO cells, and plant cells.

As described herein, it may be desirable to express a peptide of the present invention as a fusion protein. Accordingly, the invention provides fusion vectors that allow for the production of such peptides. Fusion vectors can increase the expression of a recombinant protein, increase the solubility of the recombinant protein, and aid in the purification of the protein by acting for example as a ligand for affinity purification. A proteolytic cleavage site may be introduced at the junction of the fusion moiety so that the desired peptide can ultimately be separated from the fusion moiety. Proteolytic enzymes include, but are not limited to, rTEV protease, factor Xa, thrombin, and enterokinase. Typical fusion expression vectors include, but are not limited to, pFASTBacHTb or DUAL (Invitrogen), pRS (Sikorski, et al., Genetics 122(1): 19-27 (1989)), pGEX (Smith et al., Gene 67: 31-40(1988)), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Examples of suitable inducible non-fusion E. coli expression vectors include pTrc (Amann et al., Gene 69: 301-315 (1988)) and pET 11d (Studier et al., Gene Expression Technology: Methods in Enzymology 185: 60-89 (1990)).

Recombinant protein expression can be maximized in a bacterial host by providing a genetic background wherein the host cell has an impaired capacity to proteolytically cleave the recombinant protein. (Gottesman, S., Gene Expression Technology: Methods in Enzymology 185, Academic Press, San Diego, Calif. 119-128 (1990)). Alternatively, the sequence of the nucleic acid molecule of interest can be altered to provide preferential codon usage for a specific host cell, for example E. coli. (Wada et al., Nucleic Acids Res. 20: 2111-2118 (1992)).

The inventive nucleic acid molecules can also be expressed by expression vectors that are operative in yeast. Examples of vectors for expression in yeast e.g., S. cerevisiae include pYepSec1 (Baldari, et al., EMBO J. 6: 229-234 (1987)), pMFa (Kurjan et al., Cell 30: 933-943 (1982)), pJRY88 (Schultz et al., Gene 54: 113-123 (1987)), and pYES2 (Invitrogen Corporation, San Diego, Calif.).

The nucleic acid molecules can also be expressed in insect cells using, for example, baculovirus expression vectors. Baculovirus vectors available for expression of proteins in cultured insect cells (e.g., Sf9 cells) include the pAc series (Smith et al., Mol. Cell Biol. 3: 2156-2165 (1983)), the pVL series (Lucklow et al., Virology 170: 31-39 (1989)), and pFASTBacHTb and pDUAL expression vectors (Invitrogen). In certain embodiments of the invention, the nucleic acid molecules described herein are expressed in mammalian cells using mammalian expression vectors. Examples of mammalian expression vectors include pCDM8 (Seed, B. Nature 329: 840 (1987)) and pMT2PC (Kaufman et al., EMBO J. 6: 187-195 (1987)).

The expression vectors listed herein are provided by way of example only of the well-known vectors available to those of ordinary skill in the art that would be useful to express the nucleic acid molecules. Preferred vectors include the pET24b (Novagen, Madison, Wis.), pAcSG2 (Pharmingen, San Diego, Calif.), and pFASTBacHTb (Invitrogen). The person of ordinary skill in the art would be aware of other vectors suitable for maintenance, propagation or expression of the nucleic acid molecules described herein. These are found for example in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

The invention also encompasses vectors in which the nucleic acid sequences described herein are cloned into the vector in reverse orientation, but operably linked to a regulatory sequence that permits transcription of antisense RNA. Thus, an antisense transcript can be produced to all, or to a portion, of the nucleic acid molecule sequences described herein, including both coding and non-coding regions. Expression of this antisense RNA is subject to each of the parameters described above in relation to expression of the sense RNA (regulatory sequences, constitutive or inducible expression, tissue-specific expression).

The invention also relates to recombinant host cells containing the vectors described herein. Host cells therefore include, for example, prokaryotic cells, lower eukaryotic cells such as yeast, other eukaryotic cells such as insect cells, and higher eukaryotic cells such as mammalian cells. Preferred host cells of the instant invention include E. coli and Sf9.

The recombinant host cells are prepared by introducing the vector constructs described herein into the cells by techniques readily available to the person of ordinary skill in the art. These include, but are not limited to, calcium phosphate transfection, DEAE-dextran-mediated transfection, cationic lipid-mediated transfection, electroporation, transduction, infection, lipofection, and other techniques such as those found in Sambrook, et al. (Molecular Cloning: A Laboratory Manual. 2nd, ed., Cold Spring Harbor Laboratory, Cold Spring Harbor Laboratory Press, Cold Spring Harbor, N.Y., 1989).

Host cells can contain more than one vector. Thus, different nucleotide sequences can be introduced on different vectors of the same cell. Similarly, the inventive nucleic acid molecules can be introduced either alone or with other nucleic acid molecules that are not related to the inventive nucleic acid molecules such as those providing trans-acting factors for expression vectors. When more than one vector is introduced into a cell, the vectors can be introduced independently, co-introduced or joined to the nucleic acid molecule vector.

In the case of bacteriophage and viral vectors, these can be introduced into cells as packaged or encapsulated virus by standard procedures for infection and transduction. Viral vectors can be replication-competent or replication-defective. In the case in which viral replication is defective, replication will occur in host cells providing functions that complement the defects.

Vectors generally include selectable markers that enable the selection of the subpopulation of cells that contain the recombinant vector constructs. Suitable markers include, for example, tetracycline or ampicillin-resistance genes for prokaryotic host cells and dihydrofolate reductase or neomycin resistance genes for eukaryotic host cells. However, any marker that provides selection for a phenotypic trait will be effective.

While the active protein kinases can be produced in bacteria, yeast, mammalian cells, and other cells under the control of the appropriate regulatory sequences, cell-free transcription and translation systems can also be used to produce these proteins using RNA derived from the DNA constructs described herein.

Where secretion of the peptide is desired, appropriate secretion signals are incorporated into the vector. The signal sequence can be endogenous to the proteins or heterologous to these proteins.

It is also understood that depending upon the host cell in recombinant production of the peptides described herein, the peptides can have various glycosylation patterns, depending upon the cell, or maybe non-glycosylated as when produced in bacteria. In addition, the peptides may include an initial modified methionine in some cases as a result of a host-mediated process.

The recombinant host cells expressing the peptides described herein have a variety of uses. First, the cells are useful for producing the Wee1 peptide, or a peptide that can be further purified to produce desired amounts of the peptide or fragments thereof. Thus, host cells containing expression vectors are useful for peptide production.

Host cells are also useful for conducting cell-based assays involving the protein or protein fragments. Thus, a recombinant host cell expressing a native protein is useful for assaying compounds that stimulate or inhibit protein function.

Host cells are also useful for identifying protein mutants in which the protein functions are affected. If the mutants naturally occur and give rise to a pathology, host cells containing the mutations are useful to assay compounds that have a desired effect on the mutant protein (for example, stimulating or inhibiting function) which may not be indicated by their effect on the native protein.

Genetically engineered host cells can be further used to produce non-human transgenic animals. A transgenic animal is preferably a mammal, for example a rodent, such as a rat or mouse, in which one or more of the cells of the animal include a transgene. A transgene is exogenous DNA which is integrated into the genome of a cell from which a transgenic animal develops and which remains in the genome of the mature animal in one or more cell types or tissues of the transgenic animal. These animals are useful for studying the function of the Wee1 peptide and identifying and evaluating modulators of the protein activity. Other examples of transgenic animals include non-human primates, sheep, dogs, cows, goats, chickens, amphibians, and fish.

Purification of Modified Wee1

The purification conditions and methods listed herein are provided to elucidate the approach used in the purification of the modified Wee1 peptide and for the formation, for example, of the Wee1: inhibitor complexes. Of course those of ordinary skill in the art would be aware of other purification conditions and techniques that may be suitable for the purification of the modified Wee1 proteins described herein. For examples see, Methods in Enzymology, Volume 182; Guide to Protein Purification, edited by M. P. Duetscher; Academic Press (1990).

The invention also provides a method for purifying the modified Wee1 peptide (i.e. the peptide with a poly-histidine tag and linker/cleavage), described herein, to near homogeneity from a cell culture containing the modified Wee 1 peptides and contaminant proteins other than the Wee1 peptide. The method for purifying the modified Wee1 peptide comprises subjecting the cell culture to host cell lysis, immobilized metal affinity chromatography, removal of the his-tag or other suitable tags and linker sequences at the N- or C-terminus of the modified Wee1 peptide, and size exclusion chromatography. Preferably, the method comprises subjecting the cell culture to mechanical lysis, cobalt metal affinity chromatography, histidine tag cleavage, dialysis, size exclusion chromatography, and concentration.

The modified Wee1 peptide, preferably the modified Wee1 peptide containing the N-terminal His-Tag and linker, may be purified by immobilized metal affinity chromatography (IMAC). The immobilized metal may be nickel, zinc, cobalt, or copper. Preferably, the immobilized metal is cobalt, as found in the TALON™ metal affinity resin from ClonTech. The IMAC step may be accomplished with, for example, the following resins: Ni-NTA™ resin from Qiagen, HisTrap™ resin from Pharmacia, POROS™ MC resin from Applied Biosystems or TALON resin from Clontech. Preferably, TALON resin is used. More preferably, the IMAC step is accomplished by the use of resin in a ratio of about 2 mL resin to about 1 g wet weight of whole cells, prior to cell lysis.

The IMAC step may be performed on the soluble fraction of the fermentation broth containing the expressed Wee1 peptide, which is obtained after the host cell lysis. The host cell lysis processes are known to those skilled in the art and may be selected without difficulty. For example, when E. coli is used as the host for expressing the modified Wee1 peptides, E. coli cell lysis may be enzymatically performed, for example by using lysozyme. Alternatively, mechanical processes using a french press, sonicator, or bead mill may be employed. Preferably, a mechanical lysis using a bead mill (DynoMill KDL) is used to perform E. coli cell lysis.

The cell lysis may be performed in the presence of any suitable protease inhibitors. The contaminating protease inhibiting agent may be, but is not limited to, PMSF, leupeptin, E64, EDTA, aprotinin, or a combination of inhibitors as found in Complete Protease Inhibitor tablet (Roche). Preferably, the protease inhibiting agent is 1×Complete tablet per 50 mL lysis buffer.

The Wee1-binding and contaminant removal steps of the IMAC purification may be performed in the presence of any suitable buffering agent. For example, the buffering agent may be, but is not limited to, Tris [Tris(hydroxymethyl)-aminomethane], HEPES (N-2-hydroxyethyl-piperazine-N′-2-ethanesulfonic acid), potassium phosphate, citrate-phosphate, sodium phosphate, or MOPS (3-(N-morpholino) propanesulfonic acid). Preferably, the buffering agent is about 50.0 mM Tris having a pH of about 8.0.

The binding and contaminant removal steps of the IMAC purification also may be performed in the presence of a non-ionic detergent. The detergent may be, but is not limited to, CHAPS (3-([3-cholamidopropyl]-dimethylammonio)-1-propanesulfonate), Triton X-100, Nonidet P-40, Tween 20, or Tween 80. Preferably, the detergent is about 0.5 to 1.0% w/v non-ionic nonidet P-40.

Additionally, the binding and contaminant removal steps of the IMAC purification may be performed in the presence of an ion source. The ion source may be, but is not limited to, KCl, NaCl, or sodium sulfate. Preferably, the ion source is about 0.3 M NaCl.

The elution of Wee1 from the IMAC resin may be accomplished by several modes, which are known in the art. For example, the elution of Wee1 from the IMAC column may be accomplished by using, for example, EDTA, histidine, or imidazole, or by reducing the pH. Preferably, the elution of Wee1 may be accomplished with about 80 mM imidazole.

The elution step of the IMAC purification may be performed in the presence of any suitable buffering agent(s). The buffering agent may be, but is not limited to, Tris, phosphate, HEPES, or MOPS. Preferably, the buffering agent is about 50.0 mM Tris (pH of about 8.0). The elution of Wee1 also may be performed in the presence of any suitable organic agents. The organic agent may be, but is not limited to, glycerol or ethylene glycol. Preferably, the organic agent is about 2% glycerol. The elution of Wee1 also may be performed in the presence of an ion source. The ion source may be, but is not limited to, KCl, NaCl, or sodium sulfate. Preferably, the ion source is about 50 mM NaCl.

This invention also provides a method for the removal of the his-tag or other suitable tags and linker sequences at the N- or C-terminus of the modified Wee1 peptide. The removal of the tag may be accomplished by incubation of the peptide with suitable proteases including, but not limited to, rTEV and thrombin. Preferably, the protease is a recombinant version of the Tobacco Etch Virus protease (TEV) manipulated to incorporate a poly-histidine tag at the N-terminus and used at the ratio of about 50 microg rTEV per 1 mg of tagged Wee1 peptide. The proteolytic cleavage step may be performed in the presence of suitable buffering agent(s), including, but not limited to, Tris, phosphate, HEPES, or MOPS. Preferably, the buffering agent is about 50.0 mM Tris (pH of about 8.0). Preferably, the cleavage is performed in the elution buffer from a previous IMAC purification step. In this event, the buffer may also contain imidazole, preferably at a concentration of about 80 mM. The cleavage step can also be performed in the presence of any suitable reducing agents. The reducing agent may be, but is not limited to, 2-mercaptoethanol, or TCEP (Tris[2-Carboxyethylphosphine]hydrochloride). Preferably, the reducing agent is not more than 2.0 mM 2-mercaptoethanol.

The cleavage may be performed in conjunction with a dialysis purification step to remove the cleaved tag residues and to buffer exchange. Dialysis may be performed using membranes with a molecular weight cutoff significantly below the predicted molecular weight of the modified Wee1 protein but still sufficiently large to allow the passage of low molecular weight contaminants. Preferably, a dialysis membrane with a molecular weight cutoff of about 10 kD is used. Dialysis may be performed with tied tubing or with suitably dimensioned cassettes, for example, Slide-A-Lyzer (Pierce Biotechnology, Inc.), or with any other such dialysis apparatus or method. The purification is not limited to this method and anyone competent in the art could use a number of alternative approaches. The dialysis solution may contain one or more suitable buffering agent(s) including, but not limited to, Tris, phosphate, HEPES, or MOPS. Preferably, the buffering agent is about 50.0 mM Tris (pH of about 8.0).

The dialysis of Wee1 also may be performed in the presence of any suitable organic agents. The organic agent may be, but is not limited to, glycerol or ethylene glycol. Preferably, the organic agent is about 5% glycerol. The dialysis of Wee1 also may be performed in the presence of an ion source. The ion source may be, but is not limited to, KCl, NaCl, or sodium sulfate. Preferably, an ion source is about 100 mM NaCl. The dialysis step can be performed in the presence of any suitable reducing agents. The reducing agent may be, but is not limited to, 2-mercaptoethanol, or TCEP (Tris[2-Carboxyethylphosphine]hydrochloride). Preferably, the reducing agent is not more than 2.0 mM 2-mercaptoethanol.

The invention also provides a method for removal of rTEV from the dialysed peptide sample by IMAC. The IMAC step may be accomplished, for example, with the following resins: Ni-NTA™ resin from Qiagen, HisTrap™ resin from Pharmacia, POROS™ MC resin from Applied Biosystems or TALON resin from Clontech. Preferably, TALON resin is used. More preferably, the IMAC step is accomplished by the use of TALON resin in a ratio of about 1 mL resin to about 1 mg rTEV. Removal of the rTEV from the Wee1 peptide can be performed by passing the dialysed Wee1 peptide solution over the IMAC resin, thus allowing the rTEV to bind due to its poly-histidine tag and be retained in the resin. In this event, rTEV proteolysed Wee1 peptide without tag and linker should pass through the resin (without binding) and should be retained for further purification.

The invention also provides a method for further purifying the protein using size exclusion chromatography (SEC). The SEC step may be performed using various types of chromatography resins. For example, suitable SEC resins include, but are not limited to, Sephadex™ G-100, Sephadex™ G-200, Sephacryl™ S-100, Sephacryl™ S-200, Superdex™ 75, and Superdex™ 200. Preferably, the SEC resin is Superdex™ 200.

The SEC step may performed in the presence of any suitable buffering agent(s). The buffering agent may be, but is not limited to, phosphate, HEPES, MES, Tris, bis-Tris, or bis-Tris propane. Preferably, the buffering agent is about 10.0 mM Tris (pH of about 8.0).

The SEC step may be performed in the presence of any suitable reducing agent(s). The reducing agent may be, but is not limited to, 2-mercaptoethanol, TCEP, or DTT. Preferably, the reducing agent is about 5.0 mM DTT.

The SEC step may be performed in the presence of any suitable salt(s). The salt may be, but is not limited to, NaCl, KCl, ammonium acetate, or sodium sulfate. Preferably, the salt is about 100 mM NaCl.

The SEC step may be performed in the presence of any suitable chelating agent(s). The chelating agent may be, but is not limited to, sodium citrate or EDTA. Preferably, the salt is about 1.0 mM EDTA.

The SEC step of Wee1 also may be performed in the presence of any suitable organic agents. The organic agent may be, but is not limited to, glycerol or ethylene glycol. Preferably, the organic agent is about 5% glycerol.

The invention also provides a method for forming a Wee1: inhibitor complex. The Wee1: inhibitor complex formation may be performed at any point during the Wee1 peptide purification. Preferably, the Wee1: inhibitor complex formation step is performed after the protein has been completely purified. Alternatively, the Wee1: inhibitor complex formation step may be performed prior to the SEC step. The Wee1: inhibitor complex formation step may be performed at various concentrations of Wee1. For example, the concentration of Wee1 may be in the range of about 0.02 mg/ml to about 16.0 mg/ml. Preferably, the concentration of Wee1 is about 4.0-7.0 mg/ml. Further, the Wee1: inhibitor complex formation step may be performed at various molar ratios of Wee1 to inhibitor. For example, the molar ratio of Wee1 to inhibitor may be in the range of about 1:1 to about 1:1000. Preferably, the molar ratio of Wee1 to inhibitor is about 1:2.

Crystallization of Modified Wee1 Peptide Complexes

The present invention further includes methods of using the modified Wee1 peptides of the invention to grow crystals of a Wee1 peptide: inhibitor complex. The crystallization conditions and methods listed herein are provided to elucidate one approach used in the crystallization of the Wee1 peptide: inhibitor complexes. Of course those of ordinary skill in the art would be aware of other crystallization conditions and techniques that may be suitable for the crystallization of the modified Wee1 proteins described herein. For examples see, McPherson, A., Crystallization of Biological Macromolecules, Cold Spring Harbor Laboratory Press (1999).

Generally, the crystallization comprises contacting a purified, modified Wee1 peptide with an inhibitor, wherein a stable binary complex of a Wee1 peptide: inhibitor is formed, and then growing a crystal of the Wee1 peptide: inhibitor complex by adding the solution of the binary complex to a crystallization solution. For example, in order to produce crystals of a Wee1 peptide: inhibitor complex, a solution containing at least the modified Wee1 peptide and an inhibitor (“Wee1 solution”) and a crystallization solution are provided. The concentration of the Wee1 peptide in the Wee1 solution is from about 2 mg/mL to about 16 mg/mL, preferably from about 4 mg/mL to about 8 mg/mL, and more preferably is about 6 mg/mL. The concentration of inhibitor is from about 1- and about 10-fold in excess that of the Wee1 peptide concentration, preferably, is from about 1- and about 2-fold in excess that of the Wee1 peptide concentration, and more preferably, is about 2-fold excess.

In addition to the Wee1 peptide and inhibitor, the Wee1 solution may also comprise one or more of a buffering agent, a reducing agent, a source of ionic strength, an organic agent, and/or a chelating agent. The buffering agent may be, but is not limited to, phosphate, MES, HEPES (N-2-hydroxyethyl-piperazine-N′-2-ethanesulfonic acid), Tris, bis-Tris, or bis-Tris propane. Preferably, the buffering agent is Tris of a concentration between about 10 mM and about 100 mM and about 10 mM Tris (pH 8.0). The reducing agent may be, but is not limited to, 2-mercaptoethanol, TCEP or DTT. Preferably, the reducing agent is DTT at a concentration between about 0.1 mM and about 10 mM. More preferably, the reducing agent is about 5.0 mM DTT. The Wee1 solution may contain a salt as source of ionic strength. The salt may be, but is not limited to, NaCl, KCl, ammonium acetate or sodium sulfate. Preferably, the salt is NaCl at a concentration between about 5 mM and about 500 mM. More preferably, the salt is about 100 mM NaCl. The Wee1 solution may also contain an organic agent. The organic agent may be, but is not limited to, glycerol, or ethylene glycol. Preferably, the organic agent is glycerol at a concentration between about 1% (v/v) mM and about 10% (v/v) mM. More preferably, the organic agent is about 5% (v/v) mM glycerol. Any suitable chelating agent may be used. The chelating agent preferably is 1 mM EDTA.

In addition, a crystallization solution (“Wee1 crystallization solution”) may also be used in the crystallization of Wee1 peptide: inhibitor complex. When mixed with the Wee1 solution described above, the crystallization solution preferably causes the binary complex to form well-diffracting crystals. The Wee1 crystallization solution may comprise a variety of components designed to stabilize the formation of the Wee1 peptide: inhibitor complex as a crystalline solid. For example, the crystallization solution may include, but is not limited to, a source of ionic strength, and a buffering agent. The buffering agent of the Wee1 crystallization solution may be, but is not limited to, phosphate, acetate, succinate, malonate, malate, imidazole, MES, Tris, bis-Tris propane, or any combination thereof. Preferably, the buffering agent is HEPES. The concentrations of the buffer may be in the range of about 20 mM to about 500 mM HEPES and may have a pH value between about 5.5 and about 9.0. Preferably, the buffer comprises about 100 mM HEPES and has a pH value between about 6.5 and about 8.5. Most preferably, the buffer comprises about 100 mM HEPES and has a pH of about 7.5.

The source of ionic strength of the Wee1 crystallization solution may be, but is not limited to, NaCl, KCl, ammonium sulfate, lithium sulfate, ammonium phosphate, or sodium potassium phosphate. Preferably, NaCl is added, without adjustment of the pH, to a concentration of about 3.0M to about 5.0 M, or more preferably to a concentration of about 4.0M to about 4.5 M, to a solution of the 100 mM HEPES buffer solution that has had the pH value adjusted to pH 7.5 with a 1M solution of HCl. The resulting solution preferably has an ionic strength of about 4.3 M and a final pH value in the range of about 7.0 and about 8.0.

Many possible methods known in the art may be used to grow the crystals of the Wee1 peptide: inhibitor complex, including, but is not limited to, hanging-drop vapor diffusion, sitting-drop vapor diffusion, microbatch, batch, or counter diffusion in gels or oils. Preferably, the crystallization is performed by hanging-drop vapor diffusion. When using the method of hanging drop vapor diffusion to grow the Wee1: inhibitor crystals, the Wee1 solution is mixed with a droplet of the Wee1 crystallization solution to obtain a mixed droplet solution. The mixed droplet solution is then suspended over a well of crystallization solution in a sealed container. For example, about 1 μL of the Wee1 solution is mixed with the Wee1 crystallization solution in a ratio from about 1:4 to about 4:1, and preferably from about 1:2 to about 2:1. More preferably, the ratio of the Wee1 solution to the Wee1 crystallization solution is about 1:1. In one embodiment, the mixed droplet may be suspended over a well solution containing between 0.4 mL and 1.2 mL, and more preferably about 0.5 mL, of crystallization solution. The crystallization temperature may be between about 4° C. and about 20° C., and preferably is about 18° C. The mixed droplet solution is allowed to stand suspended over the well solution containing the Wee1 crystallization solution at the temperature described above for a period of about 2 days to about 8 weeks until the Wee1 peptide: inhibitor crystals reach a size appropriate for crystallographic data collection, preferably between about 0.05×0.05×0.1 mm to about 0.3 ×0.3×0.5 mm.

Standard micro and/or macro seeding may also be used to obtain a crystal of X-ray diffraction quality, i.e. a crystal that will diffract to a resolution greater than 5.0 Å. In the preferred form, no seeding is used to grow diffraction quality crystals.

After the desired growth is achieved, the crystals of Wee1 peptide: inhibitor complex, may be harvested and bathed in a cryoprotective solution. The cryoprotective solution may comprise a variety of components designed to stabilize the formation of a vitreous solid containing the Wee1 peptide: inhibitor complex as a crystalline solid at a temperature of about 110 Kelvin. The cryoprotective solution may comprise, but is not limited to, glycerol and a diluting agent, such as, for example a solution comprising 100 mM HEPES pH 7.5 and 4.3 M NaCl. The cryoprotective solution most preferably comprises, about 85 mM HEPES pH 7.5, 3.7 M NaCl, and about 15% (v/v) glycerol.

Any inhibitor of the modified Wee1 peptide may be used. Preferably the inhibitor is selected from one of the following Wee1 kinase inhibitors:

Inhibitor name: 9-Hydroxy-4-phenyl-6H-pyrrolo[3,4-c]carbazole-1,3-dione

Chemical Structure: 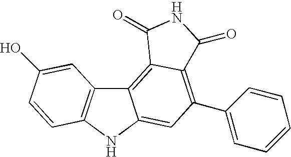
Inhibitor name: 4-(2-Chloro-phenyl)-8-(3-dimethylamino-propoxy)-9-hydroxy-6-methyl-6H-pyrrolo[3,4-c]carbazole-1,3-dione

Chemical Structure: 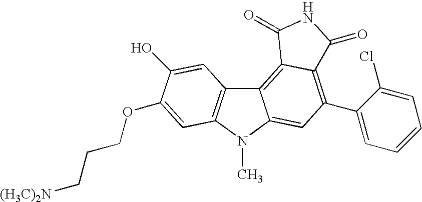
Inhibitor name: 3-(9-Hydroxy-1,3-dioxo-4-phenyl-2,3-dihydro-1H-pyrrolo[3,4-c]carbazol-6-yl)-propionic acid

Chemical Structure: 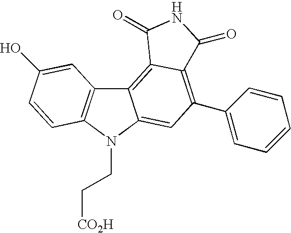
Inhibitor name: 9-Hydroxy-6-(3-hydroxy-propyl)-4-(2-methoxy-phenyl)-6H-pyrrolo[3,4-c]carbazole-1,3-dione

Chemical Structure: 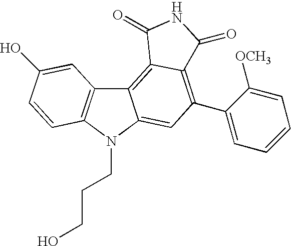
Inhibitor name: 8-(3-Amino-pyrrolidine-1-carbonyl)-4-(2-chloro-phenyl)-6-methyl-6H-pyrrolo[3,4-c]carbazole-1,3-dione

Chemical Structure:

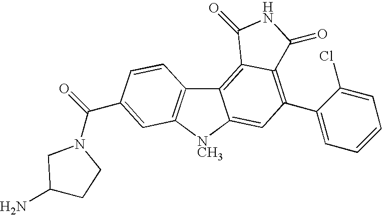

The inhibitor is present at a concentration ranging from about 1 mM to about 250 mM, and, preferably, is present at a concentration of about 4 mM to about 8 MM, even more preferably at a concentration of about 8 mM. The inhibitor is most preferably dissolved from solid in 100% dimethyl sulfoxide (DMSO).

A crystal of the present invention may take a variety of forms, all of which are included in the present invention. In one embodiment the crystals may be triclinic, monoclinic, orthorhombic, tetragonal, cubic, trigonal or hexagonal. In a preferred embodiment the crystal has tetragonal symmetry and has the space group P4₁2₁2 and has a unit cell consisting of about: a=69.78 Å, b=69.78 Å, c=157.02 Å, alpha=beta=gamma=90 degrees.

The present invention further includes methods of replacing one inhibitor within the Wee1 peptide: inhibitor crystals with another inhibitor by soaking. The soaking conditions and methods listed herein are provided to elucidate one approach used in the soaking of the Wee1 peptide: inhibitor complexes in the crystalline form. Of course those of ordinary skill in the art would be aware of other soaking conditions and techniques that may be suitable for the soaking of the Wee1 peptide: inhibitor crystals described herein.

Generally, the soaking comprises removing a Wee1 peptide: inhibitor crystal from its fully equilibrated crystallization solution and placing it in another suitable soaking solution containing the inhibitor of interest in a stable and solubilized form. The soaking solution preferably has the same components and concentration of said components as the fully equilibrated crystallization solution excluding the original inhibitor which will be replaced by the new inhibitor. The soaking solution may contain an appropriate concentration of the Wee1 peptide to solubilise the new inhibitor compound. The concentration of the new inhibitor in the soak solution may be in the range of about 1 mM to about 10 mM. Preferably, the inhibitor concentration is between about 4 mM to about 8 mM, most preferably at about 8 mM. The Wee1 peptide: inhibitor crystal may be soaked for a period of about 1 hour to an indefinite period, preferably for a period between about 12 hours and about two weeks, more preferably for a period between about 24 hours and about 1 week.

X-ray Data Collection and Structural Determination

The present invention further includes methods for collecting X-ray diffraction data on the Wee1 peptide: inhibitor crystalline complexes of the invention. The data collection conditions and methods listed herein are provided to elucidate the approach used for structural determination of the Wee1 peptide: inhibitor complexes. Of course those of ordinary skill in the art would be aware of other conditions and techniques that may be suitable for the X-ray data collection and structural determination of the modified Wee1 protein complexes described herein. For examples, see, Glusker, J., Crystal Structure Analysis for Chemists and Biologsts, Wiley-VCH Press (1994) or the International Tables for X-Ray Crystallography, Volume F, edited by M. G. Rosssman and E. Arnold, Kluwer Academic Publishers (2001).

Generally, collecting the X-ray diffraction data for the Wee1 peptide: inhibitor complex crystals comprises mounting the crystals in a cryo loop, bathing the crystals in a cryo protectant solution, rapidly cooling the crystals to about 100 K, and collecting diffraction data in the oscillation mode. The source of X-rays may be, but is not limited to, a rotating anode source such as a Rikagu RU-H3R generator, or a high energy synchrotron source such as the insertion device at beamline 17 (17-ID, IMCA-CAT) at the Argonne National Laboratory Advanced Photon Source. The preferred method of data collection is to collect an initial data set using the home source to evaluate the crystal quality and then collecting a complete data set at IMCA-CAT. The method of detecting and quantitating the diffraction data may be performed by using, for example, an image plate such as a R-Axis IV⁺⁺ from MSC/Rigaku, a charge-coupled device like the MAR-CCD X-ray detector, or a Mar345 image plate. Preferably, the resulting crystal diffracts X-rays for the determination of the atomic coordinates of the Wee1 peptide: inhibitor: complex, to a resolution of greater than 5.0 Å, more preferably to a resolution of greater than 3.0 Å, even more preferably to a resolution of greater than 2.5 Å.

Once the data is collected, it is generally corrected for Lorenz and polarization effects and converted to indexed structure factor amplitudes using data processing software, for example DENZO (Otwinowski, Z. and Minor, W., Processing of X-ray diffraction data collected in oscillation mode, Methods Enzymol. 276: 307-326 (1997)), d*Trek (Rigaku MSC) or Mosfilm (Leslie, A. G. W., Joint CCP4+ESF-EAMCB Newsletter on Protein Crystallography, No. 26 (1992)). The preferred processing software may be DENZO. The three-dimensional structure obtained from the processed diffraction data from the crystal may be determined by one or more of the following methods or by other similar methods not included in this list: Patterson difference methods, molecular replacement, multiwavelength anomalous dispersion, single-wavelength anomalous scattering, single isomorphous replacement with anomalous scattering or multiple isomorphous replacement.

Wee1 Crystallographic Structural Analysis

The present invention further includes methods for solving the three-dimensional atomic coordinates of the Wee1 peptide: inhibitor complexes using the X-ray diffraction data. The methods used for structural determination are provided to elucidate the approach used for the structural determination of the Wee1 peptide: inhibitor crystalline complexes of the invention. Those of ordinary skill in the art would be aware of other conditions and techniques that may be suitable for the X-ray structural determination of the Wee1 peptide complexes described herein. For examples see, Glusker, J., Crystal Structure Analysis for Chemists and Biologists, Wiley-VCH Press (1994) or the International Tables for X-Ray Crystallography, Volume F, edited by M. G. Rosssman and E. Arnold, Kluwer Academic Publishers (2001).

The analysis of the Wee1 template may be performed using computer modeling programs. Suitable computer modeling programs include, but are not limited to SYBYL®, GRIN/GRID®, MolCad®, GOLD®, FlexX®, QUANTA®, CharmM®, INSIGHT®, MacroModel® and ICM® (See Dunbrack et al., 1997, supra), with SYBYL® being the preferred program. Additionally, suitable computer modeling software can optionally be used to perform structural determination. Such software includes, but is not limited to, QUANTA® (Accelrys, San Diego, Calif.), CHARMm® (Accelrys), INSIGHT® (Accelrys), SYBYL® (Tripos, Inc., St. Louis), MacroModel® (Schrödinger, Inc.) and ICM (MolSoft, LLC), with SYBYL® being the most preferable program. The computer program may be used alone or combined with a docking computer program such as GRAMM (Ilya A. Vakser, Rockefeller Univ.), FlexX® (Tripos Inc.), Flexidock® (Tripos Inc.), GOLD (commercially available via Cambridge Crystallographic Data Centre, Cambridge, UK), DOCK (Irwin Kuntz, Department of Pharmaceutical Chemistry at the University of California, San Francisco), or AutoDock® (Molecular Graphics Laboratory). GOLD is most preferably used. (Jones G,; Willett P., Glen, R.; Leach, A.; Taylor, R. J. Mol. Biol. 1997, 267, 727-748. Commercially available from the Cambridge Crystallographic Data Centre). These docking computer programs scan known databases of small molecules to find core compounds that roughly fit the binding sites.

If necessary, crystallographic data in PDB (Protein DataBank) files can be “cleaned up” by modifying the atom types of the inhibitor and any water molecules that are present so that the water molecules find their lowest energy rotamer. The software may be used to add hydrogens to the PDB molecular structure file in standardized geometry with optimization of orientations of OH, SH, NH₃⁺, Met methyls, Asn and Gln sidechain amides, and His rings. Suitable software for performing this “clean up” include, but are not limited to, SYBYL®, WATCHECK (part of CCP4 suite, COLLABORATIVE COMPUTATIONAL PROJECT, No. 4, “The CCP4 Suite: Programs for Protein Crystallography.” Acta Cryst. D50, 760-763 (1994)), and REDUCE (Word, et al., “Asparagine and glutamine: using hydrogen atom contacts in the choice of side chain amide orientation” J. Mol. Bio. 285: 1733-45 (1999)), with REDUCE, or any software performing the equivalent function as REDUCE, being the most preferred software. Any suitable docking computer program may be used to further validate the refined protein structure by adding all of the hydrogens in the most favorable protonation state as well as rotating all water molecules into orientations that give the optimal interactions with the protein.

Further, the ATP substrate binding site may be characterized using, for example, GRIN/GRID® (Molecular Discovery Limited), MOLCAD® (Tripos, Inc.) contouring, CAVEAT (P. A. Bartlett, et al., CAVEAT: A program to facilitate the structure-derived design of biologically active molecules, in molecular recognition in chemical and biological problems, special Publication, Royal Chem. Soc., 78, 182-196 (1989), available from the University of California, Berkely, Calif.), GRASP (A. Nicholls, Columbia University), SitelD® (Tripos, Inc.), INSIGHT®, or SYBYL®. These softwares may be used individually or in combination. For example, the combination of GRID/GRID®, MolCad® (Tripos, Inc.) and SYBYL® is preferably used.

In the structural determination of the Wee1 kinase, it was necessary to use methods other than molecular replacement to solve the phase problem, including, the single isomorphous replacement with anomalous dispersion (“SIRAS”) technique.

Using these methods, as described above, it was discovered that the Wee1 peptide comprises an ATP substrate binding site that is defined by the three-dimensional atomic coordinates of the following amino acid residues within about 4 Å of an inhibitor located in the ATP substrate binding site: I305, G306, V313, A326, K328, E346, V360, I374, N376, E377, Y378, C379, N380, G382, F433, and D463 of SEQ ID NO: 2, or a conservatively substituted variant thereof; and by the three-dimensional atomic coordinates of the following amino acid residues within about 5 Å of an inhibitor located in the ATP substrate binding site: I305, G306, V313, A326, K328, E346, V360, I374, N376, E377, Y378, C379, N380, G381, G382, S383, D386, N431, F433, G462, D463, L464, G465 of SEQ ID NO: 2, or a conservatively substituted variant thereof.

Further, through X-ray crystallographic analysis, it has been discovered that certain features of the Wee1 peptide are responsible for the high affinity binding of the inhibitor to the Wee1 ATP binding site. This information can be used in the iterative drug design process, including, for example, modeling, design, screening and identification, and/or evaluation of chemical entities that have the potential to associate with Wee1, and thus may inhibit Wee1 activity. For example, X-ray crystallographic data has allowed for the detailed identification of Wee1's three-dimensional structure for the first time, particularly within the ATP binding domain. It is known that there are residues that are highly conserved in the kinase superfamily that are responsible for binding the ATP substrate. It has been discovered that Wee1 peptide contains a nearly invariant (in protein kinases) glycine-rich loop that is partly responsible for binding the inhibitors that interact at the ATP substrate binding site.

It has also been discovered that a Mg²⁺ cation may be present to aid in the orientation of the ATP-site binding inhibitors as well as the protein. However, in some instances the Mg²⁺ cation is not present, in which case the ATP substrate binding site may be occupied by a less tightly bound water molecule. When present, the Mg²⁺ cation is chelated in part by N431 and D463. D463 is part of a highly conserved DFG triplet in most kinases, which is actually DLG in Wee1.

Additionally, it has been discovered that the binding of inhibitors forms several interactions with the Wee1 peptide. Such inhibitors include, but are not limited to, the following carbazole compounds: 9-Hydroxy-4-phenyl-6H-pyrrolo[3,4-c]carbazole-1,3-dione; 4-(2-Chloro-phenyl)-8-(3-dimethylamino-propoxy)-9-hydroxy-6-methyl-6H-pyrrolo[3,4-c]carbazole-1,3-dione; 3-(9-Hydroxy-1,3-dioxo-4-phenyl-2,3-dihydro-1H-pyrrolo[3,4-c]carbazol-6-yl)-propionic acid; 9-Hydroxy-6-(3-hydroxy-propyl)-4-(2-methoxy-phenyl)-6H-pyrrolo[3,4-c]carbazole-1,3-dione; and/or 8-(3-Amino-pyrrolidine-1-carbonyl)-4-(2-chloro-phenyl)-6-methyl-6H-pyrrolo[3,4-c]carbazole-1,3-dione. One interaction between the Wee1 peptide and such a binding inhibitor is the filling of the deep ATP pocket by a carbazole ring or similar ring structure. The tetracyclic carbazole ring system is believed to make hydrophobic interactions with Y378, Ile 305, Val 313, A326, G382, and Phe 433. F433 is not normally found in kinases, and is believed to form a strong pi stacking interaction with the inhibitor carbazole ring systems. It is also believed that hydrogen bonds are formed between the 9-hydroxy, 1-carbonyl, 2-NH, and to a lesser extent, the 3-carbonyl of carbazole inhibitor compounds and C379, G377, and N376, respectively, of the Wee1 peptide. Another set of key interactions involves the packing of a 4-phenyl ring or similar ring structure of carbazole inhibitor compounds in a site in Wee1 formed primarily by V313, K328, N376, E346, I374, D463, and optionally tightly bound water molecules and/or a Mg ion and its associated waters. N376 and His 350 are not normally found in kinases, and together with bound waters in this pocket, impart a distinct set of interactions that strongly influence the binding affinity of inhibitors to Wee1. For example, ortho phenyl substitution generally leads to increased binding affinity against Wee1 relative to other kinases such as Chk-1. Side chains at the 6, 7, and 8-positions of carbazole inhibitor compounds are believed to form interactions with more surface-exposed residues such as Ser430, Ser383, Asn431, Gly 382, Asp386, Asn 380, Y378, Lys315, and I305 that are adjacent to the ATP substrate binding site. These interactions together with interactions with bound waters in the vicinity, can be taken advantage of to design inhibitors of Wee1. It is also possible to enhance affinity via side chains off the 4-6 positions of the carbazole inhibitor compounds that positions hydrophobic functionality between the glycine-rich loop formed by amino acids from Ser307 to Ser312 (inclusive) and the side chain of K328.

Those of skill in the art will recognize that a set of atomic coordinates for a peptide or a peptide: inhibitor complex, or a subset thereof, is a relative set of points in space that defines a complex three dimensional surface. As such it is possible to represent the same surface using an entirely different set of coordinates. Also, due to small errors in the measurement of all crystallographic data, slight variations in the individual coordinates will have little or no effect on the overall surface. Thus, an ATP substrate binding site could be generated from the atomic coordinates provided in any one of Tables 1-5 or from some variation of the atomic coordinates that still retains similar surface features, including, but not limited to, volume (both internally in cavities or in total), solvent accessibility, and surface charge and hydrophobicity. In addition, the atomic coordinates could be modified by crystallographic permutations, including, but not limited to, fractionalization, integer addition or subtraction, inversion or any combination thereof.

In addition, it would be apparent to one skilled in the art that the ATP substrate binding site described in detail above could be modified in order to obtain somewhat different three-dimensional atomic coordinates.

It should also be recognized that minor modification of any or all of the components of the peptide: inhibitor complexes that results in the generation of atomic coordinates that still retain the basic features of the three-dimensional structure should be considered part of the invention.

Computers, Computer Software, Computer Modeling

Once the atomic coordinates are known, a computer may be used for producing a three-dimensional representation of a Wee1 peptide, or a structurally related peptide. Likewise, the atomic coordinates, or related set of atomic coordinates, may be used to generate a three dimensional representation of a Wee1 peptide ATP substrate binding site or Wee1-like peptide ATP substrate binding site. Thus, another aspect of the invention involves using the atomic coordinates generated from the Wee1: inhibitor complexes as set forth in Table 1, Table 2, Table 3, Table 4, and/or Table 5, or a related set of atomic coordinates, to generate three-dimensional representations of Wee1 peptide, a structurally related peptide, or a Wee1 or Wee1-like peptide ATP substrate binding site. This is achieved through the use of commercially available software that is capable of generating three-dimensional graphical representations of molecules or portions thereof from a set of atomic coordinates.

Suitable computers are known in the art and typically include a central processing unit (CPU), and a working memory, which can be random-access memory, core memory, mass-storage memory, or a combination thereof. The CPU may encode one or more programs. Computers also typically include display, input and output devices, such as one or more display terminals, keyboards, modems, input lines and output lines. Further, computers may be networked to computer servers (the machine on which large calculations can be run in batch) and file servers (the main machine for all the centralized databases).

Machine-readable media containing data, such as the three-dimensional atomic coordinates set forth in Table 1, Table 2, Table 3, Table 4 and/or Table 5, or a related set of atomic coordinates, may be inputted using various hardware, including modems, CD-ROM drives, disk drives, or keyboards.

Machine-readable data medium can be, for example, a floppy diskette, hard disk, or an optically-readable data storage medium, which can be either read only memory, or rewritable, such as a magneto-optical disk.

Output hardware, such as a display terminal, may be used for displaying a graphical representation of the three-dimensional atomic coordinates of the Wee1 peptide as set forth in Table 1, Table 2, Table 3, Table 4, and/or Table 5, of a structurally related peptide, or of a Wee1 or Wee1-like peptide ATP substrate binding site, as described herein. Output hardware may also include a printer and disk drives.

The CPU coordinates the use of the various input and output devices, coordinates data access from storage and access to and from working memory, and determines the sequence of data processing steps. A number of programs may be used to process the machine-readable data. Such programs are discussed herein in reference to the computational methods of drug discovery.

In a preferred embodiment of the invention, atomic coordinates capable of being processed into a three-dimensional representation of a molecule or molecular complex that comprises a Wee1 or Wee1-like peptide ATP substrate binding site are stored in a machine-readable storage medium. As described below, the three-dimensional structure of a molecule or molecular complex comprising a Wee1 or Wee1-like peptide ATP substrate binding site is useful for a variety of purposes, such as in drug discovery and drug design. For example, the three-dimensional structure derived from the atomic coordinate data may be computationally evaluated for its ability to associate with chemical entities.

Wee1 Activity Inhibitors

The association of natural ligands with their corresponding binding sites on receptors or enzymes is the basis of many biological mechanisms of action. Similarly, many drugs exert their biological effects via an interaction with the binding pockets of a receptor or enzyme. An understanding of such associations can lead to the design of drugs having more favorable and specific interactions with their target receptors or enzymes, and thus, improved biological effects. Therefore, information related to ligand association with the Wee1 or Wee1-like peptide ATP substrate binding site is valuable in designing and/or identifying potential inhibitors of Wee1 peptide, or peptides structurally related thereto. Further, the more specific the design of a potential drug, the more likely that the drug will not interact with similar proteins, thus, minimizing potential side effects due to unwanted cross interactions.

The present invention provides methods of using the three-dimensional representations of the Wee1 peptide, structurally related peptides, and/or the Wee1 or Wee1-like peptide ATP substrate binding site generated from the three-dimensional atomic coordinates set forth in any of Tables 1-5, or a related set of atomic coordinates, to model the binding of candidate compounds. The methods include methods for screening and identifying potential inhibitors of Wee1 peptide, or a structurally related peptide; and for the design of or modification of chemical entities having the potential to associate with Wee1, a structurally related peptide, or a Wee1 or Wee1-like peptide ATP substrate binding site.

The compound design or modification process begins after the structure of the target, e.g., a Wee1 peptide, is resolved to of greater than 5.0 Å, preferably greater than 3.5 Å. As described above, the data generated from the resolved crystal structure is applied to a computer algorithm to generate a three-dimensional representation and, ultimately, model, of the Wee1 or structurally related peptide, and/or Wee1 or Wee1-like peptide ATP substrate binding site. Resolving the Wee1 three-dimensional structures using the X-ray crystallographic coordinates, as described above, enables one to determine whether a compound could occupy the Wee1 peptide ATP substrate binding site or a Wee1-like peptide ATP substrate binding site.

After a three-dimensional representation of the Wee1 peptide molecule, a structurally related peptide molecule, or a Wee1 or Wee1-like peptide ATP substrate binding site is generated, a chemical entity having the potential to associate with the peptide or binding site is generated by, for example, (i) assembling molecular fragments into the chemical entity; (ii) de novo design of the chemical entity; (iii) selecting a chemical entity from a small molecule database; or (iv) modifying a known inhibitor, or portion thereof, of Wee1 activity.

When designing a chemical entity, the following factors may be considered. First, the entity must be capable of physically and structurally associating with some or the entire Wee1 or Wee1-like peptide ATP substrate binding site. Second, the entity must be able to assume a conformation that allows it to associate with a Wee1 or Wee1-like peptide ATP substrate binding site directly. Although certain portions of the entity will not directly participate in these associations, those portions of the entity may still influence the overall conformation of the molecule. This, in turn, may have a significant impact on potency. Such conformational requirements include the overall three-dimensional structure and orientation of the chemical entity in relation to all or a portion of the ATP substrate binding site, and the spacing between functional groups of an entity comprising several chemical entities that directly interact with the Wee1 or Wee1-like peptide ATP substrate binding site.

The design of new compounds or the modification of known compounds may involve synthesizing or modifying compounds, or fragments thereof, via computer programs which build and link fragments or atoms into a target binding site(s) based upon steric and electrostatic complementarity, without reference to substrate analog structures. The computer program analyzes molecular structure and interactions. The computer analysis can be performed, for example, with one or more of the following computer programs: QUANTA®, CharmM®, INSIGHT®, SYBYL®, MacroModel® and ICM® (Dunbrack et al., 1997, supra), DOCK®, GRAM®, FlexX®, Flexidock®, GOLD®, AGDOCK®, or AUTO DOCK® [Dunbrack et al., 1997, supra]. SYBYL® and GOLD® are most preferably used. Selected compounds, or fragments thereof, may be positioned in a variety of orientations, or docked, within the Wee1 or Wee1-like peptide ATP substrate binding site as defined by the atomic coordinates in Tables 1-5. If compounds have been selected, then they may be assembled into a single complex. If fragments have been selected, then they may be assembled into a single compound. Assembly may be preceded by visual inspection of the relationship of the compounds or fragments to each other on the three-dimensional Wee1 or Wee1-like peptide ATP substrate binding site representation displayed on a computer screen in relation to the atomic coordinates. This visual image step may be followed by manual model building using appropriate software programs. Alternatively, compounds may be designed as a whole using either empty binding site(s) or binding site(s) containing the natural ligand(s).

Computer programs that may be used in the design or modification of the potential inhibitor include, but are not limited to, alone or in combination, QUANTA (Accelrys Inc.) and/or SYBYL® (Tripos, Inc.) and/or a docking computer program such as GOLD (commercially available via Cambridge Crystallographic Data Centre, Cambridge, UK; Jones, G., J. Mol. Biol. 245: 43-53 (1995)), FlexX (Tripos, Inc.), GRAMM (Ilya A. Vakser, Rockefeller Univ.), Flexidock (Tripos, Inc.), Dock (Ewing, T. J. A. et al., J. Comput.-Aided Mol. Des. 15: 411-428 (2001)), or AutoDock (Molecular Graphics Laboratory (Scripps Research Inst.); Goodsell, D. S., J. Mol. Recognit. 9: 1-5 (1996)). In addition, other related computer programs may be used.

The potential inhibitory or binding effect of the chemical entity on a Wee1 or Wee1-like peptide ATP substrate binding site may be analyzed prior to its actual synthesis and testing through the use of computer modeling techniques. The “modeling” includes applying an iterative or rational process to individual or multiple potential inhibitors, or fragments thereof, to evaluate their association with the Wee1 or Wee1-like peptide ATP substrate binding site and to evaluate their inhibition of Wee1 peptide activity. This procedure may include, for example, computer fitting a potential inhibitor into a Wee1 or Wee1-like peptide ATP substrate binding site to ascertain how well the shape and chemical structure of the potential inhibitor complements or interferes with the peptide. Computer programs, such as, for example, the program GOLD®, may also be used to estimate the attraction, repulsion and steric hindrance of the inhibitor to the Wee1 or Wee1-like peptide ATP substrate binding site. For example, one can screen computationally small molecule databases for chemical entities or compounds that can bind in whole, or in part, to a Wee1 or Wee1-like peptide ATP substrate binding site. In this screening, the quality of fit of such entities or compounds to the ATP substrate binding site may be judged either by shape complementarity or by estimated interaction energy (Meng, et al., J. Comp. Chem., 13:505-524 (1992)). Generally, the tighter the fit, e.g., the lower the steric hindrance and/or the greater the attractive force, the more antagonistic the potential inhibitor will be since these properties are consistent with a tighter-binding constant. If the theoretical structure, i.e., computational structure, indicates insufficient interaction and association, further testing may not be necessary. However, if computer modeling indicates a strong interaction, then the inhibitor may be synthesized and tested for its ability to bind to a Wee1 or Wee1-like peptide ATP substrate binding site. Thus, a potential inhibitor may be identified and selected, based on its computational ability to positively associate with the amino acid residues found within ATP substrate binding site.

Suitable computer programs to be used for computer modeling include, but are not limited to, QUANTA®, CharmM®, INSIGHT®, SYBYL®, MacroModel® and ICM (Dunbrack et al., 1997, supra). SYBYL® is preferably used. The computer program may be used alone or combined with a docking computer program such as DOCK®, GRAM®, FlexX®, Flexidock®, GOLD® or AUTO DOCK [Dunbrack et al., 1997, supra]. For this purpose, GOLD® is most preferably used.

The screening method and subsequent identification of potential inhibitors, may be accomplished in vivo, in vitro or ex vivo. Initial inhibitor computation analysis is optional. Instead, or additionally, high-throughput screening may be employed which may be capable of full automation at robotic workstations such that large collections of compound libraries may be screened.

In one embodiment, potential Wee1 peptide activity inhibitors may be designed and/or identified via computer programs which build and link fragments or atoms into the ATP substrate binding site based upon steric and electrostatic complimentarity, without reference to substrate analog structures. The computer program analyzes the molecular structure and interactions. The computer analysis can be performed, for example, with one or more of the following computer programs: QUANTA®, CharmM®, INSIGHT®, SYBYL®, MacroModel® and ICM® (Dunbrack et al., 1997, supra), DOCK®, GRAM®, FlexX®, Flexidock®, GOLD®, AGDOCK®, or AUTO DOCK® [Dunbrack et al., 1997, supra]. SYBYL® and GOLD® are most preferably used.

In another embodiment of the screening and identification method, the initial computer modeling is performed with one or more of the following docking computer modeling programs: Dock (Ewing, T. J. A. et al., J. Comput.-Aided Mol. Des. 15: 411-428 (2001)), AutoDock (Molecular Graphics Laboratory; Goodsell, D. S., J. Mol. Recognit. 9: 1-5 (1996)), GOLD (commercially available via Cambridge Crystallographic Data Centre, Cambridge, UK; Jones, G., J. Mol. Biol. 245: 43-53 (1995)) or FlexX (Tripos, Inc.). Potential inhibitors initially identified by the docking program(s) are elaborated using standard modeling methods as found in, for example, SYBYL® (Tripos, Inc.), QUANTA (Accelrys Inc.), INSIGHT®-II (Accelrys Inc.), GRIN/GRID (Molecular Discovery Ltd.), UNITY® (Tripos, Inc.), LigBuilder (Want, R., J. Mol. Model 6: 498-516 (2000)), or SPROUT (developed and distributed by ICAMS (Institute for Computer Applications in Molecular Sciences) at the University of Leeds, United Kingdom (Gillet, V. et al., J. Comput. Aided Mol. Design 7: 127-153 (1993))).

After a potential activity inhibitor is identified, it can either be selected from commercial libraries of compounds or alternatively the potential inhibitor may be synthesized and assayed to determine its effect(s) on the activity of Wee1, or a structurally related peptide. Optionally, the assay may be a radioactive or a non-radioactive assay, such as an ELISA. However, in a preferred embodiment, the assay is a radioactive assay.

In one embodiment of screening and identifying potential inhibitors via computer modeling, the method comprises: (a) generating a three-dimensional representation of a Wee1 peptide, a structurally related peptide, or a Wee1 or Wee1-like peptide ATP substrate binding site; (b) designing and/or building (e.g. computationally) de novo potential inhibitors; and (c) identifying the inhibitors that associate with the Wee1 or Wee 1-like ATP substrate binding site. Such inhibitors may be identified by, for example, contacting the inhibitor with a cell that expresses Wee1. A Wee1 inhibitor may be identified, for example, as a compound that inhibits the Wee1 catalyzed phosphorylation of Cdc2 at Tyr 15. The cell may be a eukaryotic cell, including, but not limited to, a yeast cell or vertebrate. Preferably, the cell is a mammalian cell. More preferably, the cell is a human cell. The protein assay can be an in vitro, in situ or in vivo, but is preferably an in vitro assay. In one such embodiment, the Wee1 catalyzed phosphorylation of Cdc2 at Tyr 15 may be determined by a cellular assay in which the Wee1 peptide is a rate-limiting factor. A measure of Cdc2 phosphorylation is determined, and a compound that inhibits that measure of phosphorylation of Cdc2 is selected as a potential drug. Examples of suitable assays that may be used for this purpose are described herein in Example 10.

In an alternative embodiment of screening and identifying potential inhibitors via computer modeling, the method comprises: (a) generating a three-dimensional representation of Wee1, a structurally related peptide, or a Wee1 or Wee1-like peptide ATP substrate binding site; (b) building (e.g. computationally) and, optionally, modifying, known potential inhibitors; and (c) identifying the inhibitors that associate with the Wee1 or Wee1-like peptide ATP substrate binding site.

In an alternative embodiment, the compound screening and identification method comprises evaluating the ability of de novo compounds to function as Wee1 activity inhibitors by, for example: (a) generating a Wee1 or Wee1-like virtual ATP substrate binding site; (b) designing (e.g. computationally) a compound structure that spatially conforms to the binding site; (c) synthesizing the compound and, optionally, analogs thereof, and (d) testing to determine whether the compound binds to the binding site.

In an alternative embodiment, the compound screening and identification method comprises evaluating the ability of known compounds to function as Wee1 activity inhibitors by, for example: (a) generating a Wee1 or Wee1-like virtual ATP substrate binding site; (b) generating (e.g. computationally) and, optionally, modifying, a known compound structure; (c) determining whether that compound spatially conforms to the binding site; (d) synthesizing the compound and, optionally, analogs thereof; and (e) testing to determine whether the compound binds to the binding site by methods described herein.

In another embodiment, wherein a potential inhibitor has been selected, the identification method comprises: (a) generating a three-dimensional representation of Wee1, or a structurally related peptide, with the potential inhibitor bound thereto; (b) modifying the potential inhibitor based on the three-dimensional representation; and (c) generating a second three-dimensional representation with the modified potential inhibitor bound thereto. Then, one can test the potential inhibitor in a biochemical assay known in the art, if desired.

In addition, when a potential inhibitor is identified, a supplemental crystal may be grown comprising the inhibitor in complex with Wee1, or a structurally related peptide, and optionally a cofactor. Molecular replacement analysis, for example, may be used to determine the three-dimensional structure of the supplemental crystal. Molecular replacement analysis may also be used in the initial crystal structure determination.

It should be understood that in all of the structure-based drug design strategies provided herein, a number of iterative cycles of any or all of the steps may be performed to optimize the selection.

Thus, according to another embodiment, the invention provides compounds that associate with a Wee1 or Wee1-like peptide ATP substrate binding site produced or identified by any one or a combination of the methods set forth above.

Wee1 Variants

As mentioned above, the present invention also provides and enables obvious variants of the amino acid sequence of the proteins of the present invention, such as naturally occurring mature forms of the proteins, allelic/sequence variants of the proteins, and non-naturally occurring recombinantly derived variants of the proteins. Such variants can be generated using techniques that are known by those skilled in the fields of recombinant nucleic acid technology and protein biochemistry. It is understood, however, that variants exclude any proteins or peptides disclosed prior to the invention.

Such variants can readily be identified/made using molecular techniques and the sequence information disclosed herein. Further, such variants can readily be distinguished from other proteins based on sequence and/or structural identity to the proteins of the present invention. The degree of identity present will be based primarily on whether the peptide is a functional variant or non-functional variant. An alternative method to using the primary sequence for describing the structural relationship between two proteins or peptides is to use the three-dimensional structures of the two related proteins. In this method, the two structures are solved by X-ray crystallography or by NMR, and then the similarity is determined by comparing the root mean square (RMS) deviation of the backbone C-alpha trace of the two species.

To determine the percent identity of two amino acid sequences or two nucleic acid sequences, the sequences are aligned for optimal comparison purposes (e.g., gaps can be introduced in one or both of a first and a second amino acid or nucleic acid sequence for optimal alignment and sequences lacking identity can be disregarded for comparison purposes). In one aspect of the invention, the length of a reference sequence aligned for comparison purposes is at least 30%, preferably 40%, more preferably 50%, even more preferably 60%. In one preferred embodiment, it is preferably at least 70%, more preferably 80%, or most preferably 90% or more of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid ‘identity’ is equivalent to amino acid or nucleic acid ‘homology’). The percent identity between the two sequences is a function of the number of identical positions shared by the sequences, taking into account the number of gaps, and the length of each gap, which need to be introduced for optimal alignment of the two sequences.

The comparison of sequences and determination of percent identity and similarity between two sequences can be accomplished using a mathematical algorithm. (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). In a preferred embodiment, the percent identity between two amino acid sequences is determined using the Needleman and Wunsch (J. Mol. Biol. (48): 444-453 (1970)) algorithm which has been incorporated into commercially available computer programs, such as GAP in the GCG software package, using either a Blossom 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences can be determined using the commercially available computer programs including the GAP program in the GCG software package (Devereux, J., et al., Nucleic Acids Res. 12(1): 387 (1984)), the NWS gap DNA CMP matrix and a gap weight of 40, 50, 60, 70, or 80 and a length weight of 1, 2, 3, 4, 5, or 6. In another embodiment, the percent identity between two amino acid or nucleotide sequences is determined using the algorithm of E. Meyers and W. Miller (CABIOS, 4: 11-17 (1989)) which has been incorporated into commercially available computer programs, such as ALIGN (version 2.0), using a PAM120 weight residue table, a gap length penalty of 12 and a gap penalty of 4.

The nucleic acid and protein sequences of the present invention can further be used as a “query sequence” to perform a search against sequence databases to, for example, identify other family members or related sequences. Such searches can be performed using commercially available search engines, such as the BLASTN and BLASTX programs (version 2.0) of Altschul, et al. (J. Mol. Biol. 215: 403-10 (1990)). BLAST nucleotide searches can be performed with the BLASTN program, score=100, word length=12 to obtain nucleotide sequences homologous to the nucleic acid molecules of the invention. BLAST protein searches can be performed with the BLASTX program, score=50, word length=3 to obtain amino acid sequences homologous to the proteins of the invention. To obtain gapped alignments for comparison purposes, Gapped BLAST can be utilized as described in Altschul et al. (Nucleic Acids Res. 25(17): 3389-3402 (1997)). When utilizing BLAST programs, the default parameters of the respective programs (e.g., BLASTX and BLASTN) can be used.

Full-length clones comprising one of the proteins of the present invention can readily be identified as having complete sequence identity to one of the kinases of the present invention as well as being encoded by the same genetic locus as the Wee1 peptide provided herein.

Allelic variants of a peptide can readily be identified as having a high degree (significant) of sequence homology/identity to at least a portion of the protein as well as being encoded by the same genetic locus as the Wee1 peptide provided herein. As used herein, two proteins (or a region of the proteins) have significant homology when the amino acid sequences are typically at least 70%, preferably at least 75%, more preferably at least 80%, and even more preferably at least 85% identical/homologous. In one preferred embodiment, it is at least 90%, or preferably at least 95% identical/homologous. A significantly homologous amino acid sequence, according to the present invention, will be encoded by a nucleic acid sequence that will hybridize to a protein encoding nucleic acid molecule under stringent conditions as more fully described above.

Non-naturally occurring variants of the Wee1 peptide of the present invention can readily be generated using recombinant techniques. Such variants include, but are not limited to deletions, additions and substitutions in the amino acid sequence of the protein. For example, one class of substitutions is conserved amino acid substitution. Such substitutions are those that substitute a given amino acid in a protein by another amino acid of like characteristics. Typically seen as conservative substitutions are the replacements, one for another, among the aliphatic amino acids Ala, Val, Leu, and Ile; interchange of the hydroxyl residues Ser and Thr; exchange of the acidic residues Asp and Glu; substitution between the amide residues Asn and Gln; exchange of the basic residues Lys and Arg; and replacements among the aromatic residues Phe; Tyr. Guidance concerning which amino acid changes are likely to be phenotypically silent are found in Bowie et al., Science 247:1306-1310 (1990).

Variants can be fully functional or can lack function in one or more activities. Fully functional variants typically contain only conservative variation or variation in non-critical residues or in non-critical regions. Functional variants can also contain substitution of similar amino acids, which result in no change or an insignificant change in function. Alternatively, such substitutions may positively or negatively affect function to some degree.

Amino acids that are essential for function can be identified by methods known in the art, such as site-directed mutagenesis or alanine-scanning mutagenesis (Cunningham et al., Science 244:1081-1085 (1989)). The latter procedure introduces single alanine mutations at every residue in the molecule. The resulting mutant molecules are then tested for biological activity such as receptor binding or in vitro proliferative activity. Sites that are critical for binding can also be determined by structural analysis such as X-ray crystallography, nuclear magnetic resonance or photoaffinity labeling (Smith et al., J. Mol. Biol. 224:899-904 (1992); de Vos et al. Science 255:306-312 (1992)).

The following examples illustrate preferred embodiments and aspects of the invention, and are intended to be non-limiting.

### EXAMPLES

### Example 1

### Wee1₂₉₁₋₅₇₅ Construct Design

Limited proteolysis and modeling experiments on a Wee1₂₉₁₋₆₀₆ construct (amino acid residues 291-606 of SEQ ID NO: 2) were used to design a Wee1₂₉₁₋₅₇₅ construct (amino acid residues 291-575 of SEQ ID NO: 2) containing a GAMG primer piece.

| | | | | | | | |
|---|---|---|---|---|---|---|---|
| 1 | msflsrqqpp | pprragaact | lrqklifspc | sdceeeeeee | eeegsghstg | edsafqepds | (SEQ ID NO: 2) |
| 61 | plpperspte | pgperrrspg | papgspgele | edlllpgacp | gadeagggae | gdsweeegfg | |
| 121 | ssspvkspaa | pyflgssfsp | vrcggpgdas | prgcgarrag | egrrsprpdh | pgtpphktfr | |
| 181 | klrlfdtpht | pksllskarg | idsssvklrg | sslfmdteks | gkrefdvrqt | pqvninpftp | |
| 241 | dslllhssgq | crrrkrtywn | dscgedmeas | dyeledetrp | akrititesn | mksryttefh | |
| 301 | elekigsgef | gsvfkcvkrl | dgciyaikrs | kkplagsvde | qnalrevyah | avlgqhshvv | |
| 361 | ryfsawaedd | hmliqneycn | ggsladaise | nyrimsyfke | aelkdlllqv | grglryihsm | |
| 421 | slvhmdikps | nifisrtsip | naaseegded | dwasnkvmfk | igdlghvtri | sspqveegds | |
| 481 | rflanevlqe | nythlpkadi | falaltvvca | agaeplprng | dqwheirqgr | lpripqvlsq | |
| 541 | eftellkvmi | hpdperrpsa | malvkhsvll | sasrksaeql | rielnaekfk | nsllqkelkk | |
| 601 | aqmakaaaee | ralftdrmat | rsttqsnrts | rligkkmnrs | vsltiy | | |

Aliquots of 7.5 μg Wee1₂₉₁₋₆₀₆ were subjected to a concentration gradient of trypsin ranging from 60 μg/mL to 0.1 μg/mL. After digesting for 1 hour at room temperature, the samples were analysed by SDS-PAGE. At trypsin concentrations below 0.9 μg/mL a stable Wee1 fragment was observed with a molecular weight loss of approximately 3 kDa. The N-terminal 6×HIS tag was still present in the fragment implying that residues had been cleaved from the C-terminal end. By correlating the known trypsin cleavage pattern with calculated molecular weight, the fragment was identified as the Wee1 sequence from residue 291 to an estimated residue 575. An overlay of seven tyrosine kinase structures and their sequence alignment with human Wee1 was downloaded from the HOMSTRAD website [http://www-cryst.bioc.cam.ac.uk/˜homstrad] (Mizuguchi et al., (1998) “HOMSTRAD: a database of protein structure alignments for homologous families.” Protein Sci., 7(11):2469-2471). Graphical display of these overlaid structures supported the results of the trypsin digest. The conserved secondary structure elements of the tyrosine kinase domains end at approximately residue 571 of full-length Wee1 as aligned and trypsin appears not to cut beyond this point leaving a stable globular protein fragment.

### Example 2

### Wee1₂₉₁₋₅₇₅ Primers for PCR Cloning

PCR primers specific for Wee1₂₉₁₋₅₇₅ were designed with restriction linkers enabling directional in frame cloning of amplicons into the pFastBacHTb vector (Invitrogen).

Primers are written 5′-3′ and have amino acid sequence as single letter code underneath. Amino and nucleic acids in italics code for the human Wee1 protein, those in normal font are generated in the cloning vectors. Arrows indicate direction of protein sequence (N to C terminal) in final expressed product.

Wee1HKFor. Human Kinase domain forward primer. Contains NcoI (CCATGG) site for cloning. M constitutes Met291 in Wee1. † indicates rTEV cleavage site.

| | | | | | | | | | | | | |
|---|---|---|---|---|---|---|---|---|---|---|---|---|
| 5′-CAG† | GGC | GCC | ATG | GGA | ATG | AAG | TCC | CGG | TAT | ACA | ACA-3′ | (SEQ ID NO: 3) |
| Q | G | A | M | G | M | K | S | R | Y | T | T | (SEQ ID NO: 4) |

—————————————————————————————————————→

Wee1HKRev1. 3′ reverse primer amplifies VLLSASRK* (reverse SEQ ID NO: 6) in human Wee1 kinase domain. Contains an EcoRI (GAATTC) site for cloning. K constitutes Lys575 in Wee1 and is followed immediately by a frame stop codon *, TGA in the transcribed sequence.

| | | | | | | | | | | | | |
|---|---|---|---|---|---|---|---|---|---|---|---|---|
| 5′-CCT | TTG | AAT | TCA | CTT | TCT | AGA | AGC | GGA | CAG | CAA | TAC-3′ | (SEQ ID NO: 5) |
| | | | * | K | R | S | A | S | L | L | V | (SEQ ID NO: 6) |

[symbol]

Human U937 cells (T-cell lymphoma) were used as a source of mRNA for template cDNA production. Amplicons using the above primers were generated from U937 cDNA via the Polymerase Chain Reaction using PlatinumPfx Taq DNA Polymerase (Invitrogen). The amplicons were purified using a High Pure PCR kit (Roche), restricted with NcoI and EcoRI and ligated into the NcoI/EcoRI sites of the pFastBacHTb vector. The resultant recombinant plasmids produced an in frame fusion of the human Wee-1 sequences with the vector His-tag sequence downstream of the polyhedrin promoter.

Ligation mixtures were transformed into the E. coli host strain DH5-α and selected on LB ampicillin media. Plasmids from amp^{r} colonies were prepared via alkaline lysis and the presence of an insert in the pFastBacHTb vector confirmed by restriction digest and PCR. Plasmids shown to contain an insert from Wee-1 specific for the kinase domain were subsequently transformed into the DH10Bac E. coli strain to generate bacmids according to the manufactures instructions (Invitrogen). A blue/white selection system allowed the identification of colonies containing bacmids with a Wee1 insertion derived from the pFastBacHTb donor vector.

Bacmids specific for Wee1 insertions were isolated from the DH10Bac hosts and introduced into primary cultures of Sf9 cells (Invitrogen) using CELLFECTIN®-mediated transfection. Cultures were grown for 72 hours at 28° C. in SF900-II media (Invitrogen) to allow expression of virus particles derived from the transfected bacmids. This primary virus stock was harvested from the culture medium and designated Wee1₂₉₁₋₅₇₅. Subsequent rounds of viral infection of Sf9 cells with Wee1₂₉₁₋₅₇₅ virus amplified the viral titre.

Wee1₂₉₁₋₅₇₅ Sequences:

Mofidied Wee1₂₉₁₋₅₇₅ sequence His-tag-predicted MW 35407.1 kDa.

| | |
|---|---|
| MSYYHHHHHHDYDIPTTENLYFQ**GAMGMKSRYTTEFHELEKIGSGEFGSVFK | (SEQ ID NO: 7) |
| CVKRLDGCIYAIKRSKKPLAGSVDEQNALREVYAHAVLGQHSHVVRYFSAWAEDDHM | |
| LIQNEYCNGGSLADAISENYRIMSYFKEAELKDLLLQVGRGLRYIHSMSLVHMDIKPSNI | |
| FISRTSIPNAASEEGDEDDWASNKVMFKIGDLGHVTRISSPQVEEGDSRFLANEVLQENY | |
| THLPKADIFALALTVVCAAGAEPLPRNGDQWHEIRQGRLPRIPQVLSQEFTELLKVMIHP | |
| DPERRPSAMALVKHSVLLSASRK* | |

“**” in SEQ ID NO: 7 indicates the rTEV cleavage site

Modified Wee1₂₉₁₋₅₇₅ with GAMG primer at 5′ end.

Protein sequence His-tag predicted MW 32438.9 kDa:

| | |
|---|---|
| GAMGMKSRYTTEFHELEKIGSGEFGSVFKCVKRLDGCIYAIKRSKKPLAGSVDE | (SEQ ID NO: 8) |
| QNALREVYAHAVLGQHSHVVRYFSAWAEDDHMLIQNEYCNGGSLADAISENYRIMSY | |
| FKEAELKDLLLQVGRGLRYIHSMSLVHMDIKPSNIFISRTSIPNAASEEGDEDDWASNKV | |
| MFKIGDLGHVTRISSPQVEEGDSRFLANEVLQENYTHLPKADIFALALTVVCAAGAEPLP | |
| RNGDQWHEIRQGRLPRIPQVLSQEFTELLKVMIHPDPERRPSAMALVKHSVLLSASRK* | |

### Example 3

### Expression & Purification of Wee1₂₉₁₋₅₇₅

Construct Wee1₂₉₁₋₅₇₅ (SEQ ID NO:7) was expressed in a culture of log phase Sf9 cells in Sf900-II media. Two 250 ml cultures in 1 L flasks were infected at a dilution of 1:1250 (volume viral stock:volume culture) after the sf9 cells had grown to 1.2×10⁶ cells/ml. The appropriate dilution was determined empirically in small scale cultures as that giving ˜2-3 fold increase in cell count over 66 hours and giving the greatest protein yield as visualised in SDS-PAGE gels. Cultures were harvested 66 hours post-infection and centrifuged at 1000×g for 15 minutes at 4° C. The pellet was resuspended in buffer 1 (PBS with 1×Complete EDTA-free tablet (Roche) per 50 mL) and centrifuged at 1000×g for 10 minutes at 5-10° C. The pellet was gently resuspended in an equal volume of buffer 1 before being beaded into liquid nitrogen for storage at −80° C. If purification is to proceed immediately after cell harvest, the PBS/freezing steps can be eliminated and the cells can instead be resuspended in buffer 2 below and homogenised as normal.

Approximately 10 g of Sf9 beads were thawed by mixing with 25 ml buffer 2 (50 mM Tris pH 8.0 with 1×Complete EDTA-free tablet per 50 ml). Thawed cells were homogenised in a glass dounce homogeniser. The lysate was centrifuged at 12,000×g for 10 minutes at 4° C. The cleared lysate was passed over a buffer 2 (50 mM Tris pH 8.0 containing protease inhibitors) pre-equilibrated Talon (CLONTECH) column by gravity flow. The resin was washed repeatedly with buffer 2 and then buffer 3 (50 mM Tris pH 8.0) before eluting the his-tagged Wee1₂₉₁₋₅₇₅ protein with buffer 4 (80 mM imidazole, 50 mM Tris pH 8.0, 50 mM NaCl, 2% (v/v) glycerol). The eluted protein was quantified by Bradford assay, mixed with rTEV (75 μg rTEV/mg Wee1₂₉₁₋₅₇₅), and dialysed overnight at 4° C. into buffer 5 (50 mM Tris pH 8.0, 100 mM NaCl, 2 mM beta-mercaptoethanol, 5% (v/v) glycerol).

The cleaved Wee1₂₉₁₋₅₇₅ was passed over a second Talon column (2 ml Talon resin) to remove uncleaved protein and the his-tagged rTEV. The protein solution was concentrated (using a Centricon MW cut-off 10K) and passed through a Superdex 200 HR 10/30 column (Pharmacia) equilibrated with buffer 6 (10 mM Tris pH 8.0, 100 mM NaCl, 1 mM EDTA, 5 mM dithiothreitol, 5% (v/v) glycerol). The central fractions of the protein peak were pooled (to avoid impurities and non-homogeneity) and concentrated to ˜7 mg/ml before 50 μl aliquots were flash frozen in liquid nitrogen for storage at −80° C.

### Example 4

### Crystallization of Wee1₂₉₁₋₅₇₅: 9-Hydroxy-4-phenyl-6H-pyrrolo[3,4-c]carbazole-1,3-dione Complex

A complex of Wee1₂₉₁₋₅₇₅ peptide with bound inhibitor was prepared by mixing a 50 μl aliquot of protein with 2.5 μl 4 mM 9-hydroxy-4-phenyl-6H-pyrrolo[3,4-c]carbazole-1,3-dione (in DMSO). The complex was diluted with buffer 6 to 5-6 mg/ml and incubated on ice for 15 minutes before centrifuging at 13,000 rpm for 5 minutes at 4° C. Crystals were grown in 2-4 μl hanging drops by vapour diffusion, the drops suspended over 0.5 ml reservoir solution (100 mM HEPES pH 7.5, 4.3 M NaCl). Hanging drops were formed by mixing equal volumes of protein solution and reservoir solution. Crystals appeared within 2-3 days and grew to maximum size (up to 0.5 mm in longest dimension) within 2-3 weeks. Crystals were placed in cryoprotectant solution (85 μl reservoir solution with 15 μl glycerol) for less than 1 second, looped in nylon loops (Hampton Research) and flash-frozen in liquid nitrogen.

### Example 5

### Crystallographic Data Collection

Crystals from Example 4 were shipped in a dry shipper to the APS synchrotron facility in Argonne, Ill. for data collection. Data were collected at −170° C. at the undulator beam line 17-ID as summarized in Table 6. This station was equipped with cryogenically cooled Si(111) 2-crystal monochromator, Pt/Pd-coated ULE mirror, and 165 mm Mar Research CCD. Data were processed with the Denzo/HKL package (Otwinowski et al., (1997) “Processing of X-ray Diffraction Data Collected in Oscillation Mode” Methods in Enzymology, Volume 276: Macromolecular Crystallography, part A, p.307-326, C. W. Carter, Jr. & R. M. Sweet, Eds., Academic Press) to 1.8 Å resolution. 2.25 Å resolution native data were also collected at −160° C. using Cu K-α radiation from a Rigaku rotating anode generator equipped with Supper-like total reflection mirrors and Mar345 image-plate detector. The 2.25 Å data set was used in structure determination.

### Example 6

### Structural Determination of the Initial Wee1₂₉₁₋₅₇₅: Inhibitor Complex Structure

Using the data from Example 5, heavy atom compounds were screened for Wee1₂₉₁₋₅₇₅ (without his-tag) binding in solution using native PAGE for visualisation. Gel shifts relative to native protein indicate successful heavy atom binding while aggregated and/or precipitated proteins will not enter the gel. K₂Pt(NO₂)₄, HgCl₂, and Hg(CN)₂ soaks all showed gel shifts.

The structure was solved by single isomorphous replacement (SIR) from a crystal soaked in 1 mM HgCl₂ for 10 minutes. The soak solution was formed by adding HgCl₂ directly to the drop containing the crystal to the required 1 mM concentration. The derivative crystal was soaked in cryoprotectant (no HgCl₂), flash-frozen, and 2.6 Å data collected on the in-house rotating anode machine as described above for native crystals.

Three mercury atoms were identified and their locations, thermal parameters, and occupancy, refined using SOLVE (Terwilliger et al., (1999) “Automated structure solution for MIR and MAD” Acta Cryst. D55, 849-861). The SOLVE phases were input to RESOLVE (Terwilliger, T. C. (1999) “Reciprocal-space solvent flattening” Acta Crystallographica D55, 1863-1871; Terwilliger, T. C. (2000) “Maximum likelihood density modification” Acta Cryst. D56, 965-972). Details of the SIR calculations are listed in Table 7. The RESOLVE map was input to MAID (Levitt, D. G. (2001) “A New Software Routine that Automates the Fitting of Protein X-Ray Crystallographic Electron Density Maps” Acta Cryst. D57, 1013-1019) for automated model building. MAID effectively built a backbone trace for approximately 90% of the secondary structure elements. A homology model was overlaid on the MAID trace and manually built into the experimental electron density using the program (Jones et al., (1991) “Improved methods for the building of protein models in electron density maps and the location of errors in these models” Acta Cryst. A47 110-119).

### Example 7

### Refinement of the Initial Wee1₂₉₁₋₅₇₅: Inhibitor Complex Structure

The structure from Example 6 was refined to 1.81 Å resolution using CNS (Brünger, A. T., Adams, P. D., Clore, G. M., DeLano, W. L., Gros, P., Grosse-Kunstleve, R.W., Jiang, J- S., Kuszewski, J., Nilges, M., Pannu, N. S., Read, R. J., Rice, L. M., Simonson, T., and Warren, G. L. (1998). Crystallography & NMR System: A New Software Suite for Macromolecular Structure Determination. Acta Cryst. D54, 905-921) initially by rigid body refinement of the whole protein model and then subsequently isolating individual secondary structure elements. Iterative cycles of manual rebuilding, simulated annealing, and conjugate gradient minimization, located 87% of the main chain sequence and 86% of the side chains. Throughout, 2fo-fc maps (including the experimental phases) and the SIR map (experimental phases only) were used for model building. In addition, visualisation of CNS density modified maps (solvent flipping) and composite omit maps aided the building process. All maps showed clear density for the inhibitor allowing manual docking and positional refinement. Water molecules and metal ions were automatically built after the protein/inhibitor model was refined by individual B-factor refinement in CNS. Details of the CNS refinement are listed in Table 8. Final refinement used REFMAC (Murshudov et al. (1997) “Refinement of Macromolecular Structures by the Maximum-Likelihood Method” Acta Cryst. D53, 240-255). TLS and CGMAT refinement and eased restraints (Table 8). The current X-ray model contains Wee1 amino acid residues 291-435, 456-569 from SEQ ID NO: 2, inhibitor: 9-Hydroxy-4-phenyl-6H-pyrrolo[3,4-c]carbazole-1,3-dione, 143 waters, and 2 magnesium ions (identified by octahedral coordination geometry consistent with metal binding). Side chains for residues E309, F310, K331, L334, and Q474 were modeled as alanine. The N-terminal residues GAMG (from the cloning site) (amino acid residues 1-4 from SEQ ID NO: 8) and the C-terminal residues 570-LSASRK-575 (amino acid residues 570-575 of SEQ ID NO: 2) were not located in the crystal structure.

### Example 8

### Crystal Structures of Wee1₂₉₁₋₅₇₅: Inhibitor Complexes

Additional crystal structures were obtained of Wee1₂₉₁₋₅₇₅ in complex with the following inhibitors:
4-(2-Chloro-phenyl)-8-(3-dimethylamino-propoxy)-9-hydroxy-6-methyl-6H-pyrrolo[3,4-c]carbazole-1,3-dione;
3-(9-Hydroxy-1,3-dioxo-4-phenyl-2,3-dihydro-1H-pyrrolo[3,4-c]carbazol-6-yl)-propionic acid;
9-Hydroxy-6-(3-hydroxy-propyl)-4-(2-methoxy-phenyl)-6H-pyrrolo[3,4-c]carbazole-1,3-dione; and
8-(3-Amino-pyrrolidine-1-carbonyl)-4-(2-chloro-phenyl)-6-methyl-6H-pyrrolo[3,4-c]carbazole-1,3-dione.

In each case, crystals were grown using methods similar to that set forth in Example 4, and data (set forth in Table 9) were collected using methods similar to that set forth in Example 5, either at IMCA-CAT or in Auckland. The structures were determined by molecular replacement using the initial Wee1₂₉₁₋₅₇₅: 9-Hydroxy-4-phenyl-6H-pyrrolo[3,4-c]carbazole-1,3-dione crystal structure (with waters, metal ions, and inhibitor removed) as a start model, and using CNS. Essentially, this involved only a single round of rigid body minimization followed by the standard refinement methods detailed in Example 7. Final refinement of the structures used either CNS or REFMAC as indicated in Table 9.

### Example 9

### Design of Wee1 Inhibitors Using Structural Information

Knowledge of the detailed protein environment around an inhibitor such as 9-hydroxy-4-phenyl-6H-pyrrolo[3,4-c]carbazole-1,3-dione (IC₅₀=100 M against the protein) allows one to use the molecular modeling techniques described herein to design analogs containing groups that are predicted to pick up additional steric (i.e. packing) and hydrogen bonding interactions with the amino acid residues of, and/or tightly bound water molecules, and/or a magnesium cation and its associated waters that comprise the ATP substrate binding site, and thus display increased potency.

a. Design of 9-Hydroxy-6-(3-hydroxy-propyl)-4-(2-methoxy-phenyl)-6H-pyrrolo[3,4-c]carbazole-1,3-dione Wee1 Inhibitor

For example, using the knowledge set forth above regarding the detailed Wee1 inhibitor binding protein environment around the 9-hydroxy-4-phenyl-6H-pyrrolo[3,4-c]carbazole-1,3-dione inhibitor, a 9-hydroxy-6-(3-hydroxy-propyl)-4-(2-methoxy-phenyl)-6H-pyrrolo[3,4-c]carbazole-1,3-dione Wee1 inhibitor was designed. The terminal hydroxy group on the N6-sidechain was predicted via modeling to pick up interactions with Ser383 and Ser430 of the protein (in contrast 9-hydroxy-4-phenyl-6H-pyrrolo[3,4-c]carbazole-1,3-dione is unsubstituted at the N-6 position). Additionally, the 2-OCH₃ substituent on the 4-phenyl ring in this inhibitor was predicted to form additional packing interactions in the vicinity of I374, A326, K328, Ile327, and N376 of the protein (in contrast 9-hydroxy-4-phenyl-6H-pyrrolo[3,4-c]carbazole-1,3-dione contains no phenyl substitution).

As described in Example 8, Wee1₂₉₁₋₅₇₅: 9-Hydroxy-6-(3-hydroxy-propyl)-4-(2-methoxy-phenyl)-6H-pyrrolo[3,4-c]carbazole-1,3-dione crystals were grown and the crystalline structure thereof determined. The resulting crystalline structure confirmed these interactions were present. Moreover, Wee1 activity assays demonstrated an improved inhibitory activity of IC₅₀=30 nM vs Wee1.

b. Design of 3-(9-Hydroxy-1,3-dioxo-4-phenyl-2,3-dihydro-1H-pyrrolo[3,4-C]carbazol-6-yl)-propionic acid Wee1 Inhibitor

The knowledge set forth above regarding the detailed Wee1 inhibitor binding protein environment around the 9-hydroxy-4-phenyl-6H-pyrrolo[3,4-c]carbazole-1,3-dione inhibitor, was also used to design the 3-(9-Hydroxy-1,3-dioxo-4-phenyl-2,3-dihydro-1H-pyrrolo[3,4-c]carbazol-6-yl)-propionic acid Wee1 inhibitor. The propionic acid side chain at the 6 position was predicted via modeling to pick up interactions with Ser383 and Ser430 (which it does through bound water molecules), as well as impart selectivity for Wee1 against other kinases, such as, for example, Chk1, which contain acidic Glu residues at the corresponding positions in the protein. These Glu residues which, would be expected to electrostatitically repulse the side chain carboxylate, lead to diminished potency vs Chk1.

As described in Example 8, Wee1₂₉₁₋₅₇₅: 3-(9-Hydroxy-1,3-dioxo-4-phenyl-2,3-dihydro-1H-pyrrolo[3,4-c]carbazol-6-yl)-propionic acid crystals were grown and the crystalline structure thereof determined. The predicted selectivity was confirmed; while 9-Hydroxy-4-phenyl-6H-pyrrolo[3,4-c]carbazole-1,3-dione is only 1.6-fold selective for Wee1 over Chk1, 3-(9-Hydroxy-1,3-dioxo-4-phenyl-2,3-dihydro-1H-pyrrolo[3,4-c]carbazol-6-yl)-propionic acid displayed 18.6-fold selectivity for Wee1 over Chk1. Wee1 activity assays demonstrated inhibitory activity of IC₅₀=22 nM vs Wee1.

### Example 10

### In vivo and in vitro Wee1 Activity Assays

The phosphorylation state of the physiologic substrate of Wee1 (tyrosine 15 on Cdc2 kinase) may be measured by, e.g., western blot procedures. This is accomplished by means of a phosphospecific antibody whose signal is normalized by comparison to the total amount of Cdc2 detected in the samples.

a. Procedure for detecting phosphotyrosine 15, and total Cdc-2 from cultured HT-29 cells in response to potential check point abrogators in cells treated with or without. Adriamycin and/or Nocodazole

HT-29 cells were grown in Dulbecco™s Modified Eagle Medium with high glucose, supplemented with 1 mM sodium pyruvate, 2 mM L-glutamine, 16 mM HEPES, 8 mM MOPS, and 10% fetal bovine serum. The cells were incubated at 37° C., in 5% CO₂, and 100% relative humidity. Cells were grown and treated in 6-well tissue culture plates. Cells were seeded in 3 mL media at a concentration of 200,000 cells per mL. Once seeded the cells were allowed to attach for 24 hours. All treatments were done in duplicate wells. The wells that were treated with Adriamycin (ADR) were exposed to 1 μM ADR for 1 hour. ADR was dissolved in sterile distilled water. After the 1 hour incubation, the cells were washed twice with 2 ml media and then incubated in 3 ml media for 16 hours. After the 16-hour incubation, the cells were treated with various concentrations of abrogator and with or without Nocodazole (NOC) at 50 ng/ml. Abrogators were dissolved in dimethylsulfoxide (DMSO) at a concentration of 10 mM and diluted with growth medium before being added to the cells and NOC was dissolved at 1 mg/mL in DMSO and diluted with growth medium before administration to the cells. The cells were incubated for 6 hours with the abrogator and NOC. The duplicate wells were scraped, on ice, and combined in a 15 ml centrifuge tube. The wells were rinsed with Dulbecco™s phosphate buffered saline (DPBS) without calcium and magnesium and the rinse combined with the scraped cells. The cells were centrifuged at 200×g at 4° C. for 5 minutes. The supernatant was discarded and the pellets resuspended in 100 μL DPBS. The cell suspension was then transferred to 1.5 ml eppendorf centrifuge tubes and centrifuged at 4° C. for 4 minutes at 4000 rpm. After the supernatant liquid was removed, the pellet was frozen on dry ice and stored at −80° C. The pellets were thawed on ice prior to lysis. The lysis buffer, ELB (2.5 mM HEPES (7.5), 150 mM NaCl, 25 μM NaF and 0.5% NP40 supplemented with 1 mM AEBSF, 1 mM sodium orthovanadate, and 1 mM dithiothretol, and 5 complete protease inhibitor cocktail tablets (Roche Biochemicals). The tablets were dissolved in 2 mL distilled water and diluted 1:25 in the lysis buffer. The pellets were suspended in 100 μl complete lysis buffer and incubated on ice for 30 minutes. Following lysis, the suspension was centrifuged at 14,000 rpm for 15 minutes at 4° C. The supernatant liquid was collected and the protein concentration determined using the Pierce BCA protein Assay Kit per manufacturers instructions. The protein concentration was adjusted to 3 mg/mL with DPBS. The samples were then diluted 1:1 with Invitrogen Tris-glycine sample buffer supplemented with 50 μl/ml 2-mercaptoethanol, boiled for 3 minutes, and stored frozen at −20° C.

Thirty micrograms of protein per lane were run on Novex pre-cast 12%, 1.5 mm, 10-well, tris-glycine polyacrylimide gels using Novex running buffer and Invitrogen (See Blue Plus 2 molecular weight standards). The gels were run at 100 volts for 30 minutes then 125 volts for 1.5 hours. The proteins were transferred to 0.45 μm pore nitrocellulose membranes using Novex transfer buffer 20 and the Novex X-Cell II blot module. The nitrocellulose membranes were blocked overnight at room temperature. The blocking buffer was 5 mM Tris (8.0), 150 mM NaCl, 0.1% Tween 20, 1 mM NaF, 10 mM glycerolphosphate, 100 μM sodium orthovanadate, and 3% bovine serum albumen. After blocking, the gel was treated with biotinylated antiphosphotyrosine antibody, diluted 1:5000 in blocking buffer. The membranes were incubated for 2 hours at room temperature with constant rocking. The antibody solutions were removed and the membranes were washed 3 times for 20 minutes each with TNT buffer. TNT buffer consisted of 50 mM Tris (8.0), 150 mM NaCl, and 0.1% Tween 20.

Secondary antibody was then added in blocking buffer. Neutravidin HRP at 1:40,000 was used for the biotinylated phosphotyrosine 15 blots. The blots remained in secondary antibody for 1 hour at room temperature followed by three 20-minute washes with TNT buffer. Protein bands were detected using the Amersham Pharmacia ECL detection kit and Kodak Bio Max film per manufacturer's instructions.

The phosphotyrosine 15 membranes were stripped using the Chemicon International Re-Blot kit per manufacturer's instructions. The blots were then washed twice with TNT buffer and once with blocking buffer for 20 minutes each.

Anti Cdc-2 (cdk1; Labvision Corporation) were diluted 150 μl per 50 ml blocking buffer and incubated with the blots for 2 hours at room temperature followed by three 20-minute washes in TNT buffer. The secondary antibody was Bio Rad goat antimouse HRP and was diluted 1:10,000 in blocking buffer before a 1 hour incubation with the blots at room temperature. Three 20-minute washes preceded ECL detection.

b. Wee1 Inhibition Filter Binding Assay (OY) to Test for Wee1 Inhibition Activity

The Wee1 kinase assay measures enzyme mediated phosphorylation of tyrosine on a synthetic peptide substrate in the presence of compounds being tested. The assay was carried out in 96 well filter microtiter plates (Millipore #MADP NOB10). Compounds were dissolved and diluted in DMSO. 10 μl 3×EDB buffer (150 mM Tris, pH 8.0, 30 mM NaCl, 30 mM MgCl₂, 3 mM DTT), 18 μl water, and 2 μl of drug dilution were added to the test wells and mixed thoroughly. Ten (10) μl of enzyme-substrate mixture was added to the wells. The Wee1 enzyme (human Wee1 kinase aa215-647, Onyx Pharmaceuticals, expressed in and purified from a baculovirus protein expression system) concentration was 0.01 μg/μl and the substrate (Poly Omithine:Tyrosine (4:1), Sigma Chemical Co.) was 0.6 μg/μl in 1×EDB buffer. The plates were mixed thoroughly for 5 minutes at room temperature. The reaction was started by adding 10 μl of 1×EDB buffer containing 47.5 μM ATP (Sigma) and 0.026 μCi/μl γ-³²P-ATP (ICN Biomedicals, Inc.) The plates were mixed at room temperature for 20 minutes. The reaction was stopped by adding 50 μl of ice cold 20% TCA with 0.1M tetrasodium pyro-phosphate. Plates were incubated on ice or refrigerated at 4° C. for 1 hour. Liquid reaction mixture was removed on a vacuum manifold and the precipitated phosphorylated substrate was rinsed 5 times with 200 μl ice cold 10% TCA with 0.1M tetrasodium pyrophosphate. 25 μl liquid scintillation cocktail were added to the membrane bound substrate and the plate read in a Microbeta (Perkin-Elmer) plate reader. Activity of compounds was calculated in comparison to uninhibited control determinations in each assay.

All references cited herein are incorporated by reference in their entirety.

While the invention has been described in conjunction with examples thereof, it is to be understood that the foregoing description is exemplary and explanatory in nature, and is intended to illustrate the invention and its preferred embodiments. Through routine experimentation, the artisan will recognize apparent modifications and variations that may be made without departing from the spirit of the invention. Thus, the invention is intended to be defined not by the above description, but by the following claims and their equivalents.

### Tables 6-9

TABLE 6
Summary of Data Collection-Wee1₂₉₁₋₅₇₅: Inhibitor 1* Complex

| | Native IMCA-CAT | Native | HgCl₂ Derivative |
|---|---|---|---|
| | 17-ID | Rotating Anode | Rotating Anode |
| Space Group | P4₁2₁2 | P4₁2₁2 | P4₁2₁2 |
| Unit Cell (Å) | 69.78, 69.78, 157.02 | 69.62, 69.62, 157.05 | 69.37, 69.37, 156.77 |
| Resolution (Å) | 1.81 | 2.25 | 2.6 |
| Total Reflections | 734,175 | 189,188 | 750,884 |
| Unique Reflections | 34,834 | 19,169 | 12,493 |
| Completeness % (shell) | 95.3 (92.3) | 94.9 (77.4) | 99.8 (99.2) |
| Av. I/σ | 23 (2.6) | 25 (4.9) | 45 (13) |
| R_{sym} (shell) | 0.082 (0.362) | 0.079 (0.401) | 0.081 (0.275) |

*Inhibitor 1: 9-Hydroxy-4-phenyl-6H-pyrrolo[3,4-c]carbazole-1,3-dione

TABLE 7
Summary of SOLVE/RESOLVE SIR Calculations -
Wee1₂₉₁₋₅₇₅: Inhibitor 1* Complex

| SIR: | | | | |
|---|---|---|---|---|
| Resolution | 3.0 | | | |
| # Measurements | 7454 | | | |
| Rᵢₛₒ | 0.157 | | | |
| Rₐₙₒ | 0.026 | | | |
| | x, y, z | occupancy | B-factor | Height I/σ |
| SOLVE: | | | | |
| Hg Peak 1 | 0.045, 0.544, 0.116 | 0.125 | 25.5 | 10.7 |
| Hg Peak 2 | 0.279, 0.437, 0.056 | 0.192 | 49.5 | 14.0 |
| Hg Peak 3 | 0.308, 0.299, 0.068 | 0.128 | 60.0 | 10.2 |
| FOM | 0.32 | | | |
| Z-score | 12.07 | | | |
| RESOLVE: | | | | |
| FOM | 0.63 | | | |
| Fraction from | 0.36 | | | |
| SOLVE | | | | |
| Fraction from | 0.64 | | | |
| Map | | | | |

*Inhibitor 1: 9-Hydroxy-4-phenyl-6H-pyrrolo[3,4-c]carbazole-1,3-dione

TABLE 8
Refinement Statistics - Wee1₂₉₁₋₅₇₅: Inhibitor 1* Complex

| | CNS | REFMAC |
|---|---|---|
| Resolution Range | 500-1.81 | 47.14-1.81 |
| Reflections (F > 0.0) | 34,674 | 33,050 |
| Total Non-H Atoms | 2213 | 2213 |
| R (working set, 95%) | 0.251 | 0.218 |
| R_{f} (free, 5%) | 0.268 | 0.237 |
| Fit in Highest Shell: | | |
| Resolution Range | | 1.806-1.853 |
| Reflections (F > 0.0) | | 2240 |
| R (working, 95%) | | 0.256 |
| R_{f} (free, 55) | | 0.251 |
| Estimated Coordinate Error: | | |
| ESU based on R | | 0.121 |
| ESU based on R_{f} | | 0.113 |
| ESU based on max. likelihood | | 0.075 |
| ESU for B values based on max. likelihood | | 2.389 |
| Correlation Coefficient Fo-Fc | | 0.939 |
| RMSD deviations: | | |
| Bond lengths (Å) | | 0.022 |
| Bond angles (°) | | 2.099 |
| Chiral centre restraints (Å) | | 0.205 |
| Planarity restraints (Å) | | 0.010 |
| Average B-factors: (Å²) | | |
| Main Chain (max) | | 24.9 (56.5) |
| Side Chain (max) | | 28.7 (60.0) |
| Inhibitor (max) | | 23.4 (29.2) |
| Magnesium (max) | | 35.5 (37.7) |
| Water (max) | | 31.2 (56.1) |
| Ramachandran Plot: (%) | | |
| Most Favoured regions | | 91.2 |
| Additional Allowed | | 7.9 |
| Generously Allowed | | 0.9 |
| Disallowed | | 0.0 |

*Inhibitor 1: 9-Hydroxy-4-phenyl-6H-pyrrolo[3,4-c]carbazole-1,3-dione

TABLE 9
Summary of Crystallographic Details of Wee1₂₉₁₋₅₇₅ Structures
Containing Inhibitors 2-5 in Space Group P4₁2₁2

| | Inhibitor 2* | Inhibitor 2* | | | |
|---|---|---|---|---|---|
| | Conformation 1 | Conformation 2 | Inhibitor 3* | Inhibitor 4* | Inhibitor 5* |
| X-ray source | IMCA-CAT | Auckland | IMCA-CAT | IMCA-CAT | Auckland |
| | | Rotating Anode | | | Rotating Anode |
| Unit cell (Å) | 68.86, 68.86, | 68.86, 68.86, | 69.47, 69.47, | 69.93, 69.93, | 69.26, 69.26, |
| | 157.74 | 156.24 | 157.34 | 157.92 | 157.27 |
| Resolution (Å) | 2.0 | 2.2 | 1.9 | 2.2 | 2.2 |
| Total reflections | 92,973 | 220,859 | 227,921 | 196,025 | 232,958 |
| Unique | 23,921 | 19,658 | 29,232 | 20,628 | 20,263 |
| reflections | | | | | |
| Completeness % | 90.7 (93.6) | 98.7 (87.4) | 92.7 (71.9) | 99.1 (99.7) | 99.9 (100) |
| (shell) | | | | | |
| Av. I/σ (shell) | 13 (3.3) | 33 (4.2) | 24 (2.1) | 26 (3.8) | 33 (5.8) |
| Rsym (shell) | 0.118 (0.308) | 0.073 (0.405) | 0.088 (0.329) | 0.090 (0.507) | 0.084 (0.573) |
| Refinement | REFMAC | REFMAC | CNS | CNS | REFMAC |
| program | | | | | |
| Reflections | 22,683 | 18,599 | 28,996 | 20,489 | 19,180 |
| Total non-H | 2115 | 2152 | 2152 | 2041 | 2185 |
| atoms | | | | | |
| R (working set, | 24.8 | 20.4 | 28.6 | 26.5 | 20.7 |
| 95%) | | | | | |
| R_{f}(free, 5%) | 30.2 | 24.3 | 30.7 | 28.8 | 22.5 |

*Inhibitor 2: 4-(2-Chloro-phenyl)-8-(3-dimethylamino-propoxy)-9-hydroxy-6-methyl-6H-pyrrolo[3,4-c]carbazole-1,3-dione;
Inhibitor 3: 3-(9-Hydroxy-1,3-dioxo-4-phenyl-2,3-dihydro-1H-pyrrolo[3,4-c]carbazol-6-yl)-propionic acid;
Inhibitor 4: 9-Hydroxy-6-(3-hydroxy-propyl)-4-(2-methoxy-phenyl)-6H-pyrrolo[3,4-c]carbazole-1,3-dione; and
Inhibitor 5: 8-(3-Amino-pyrrolidine-1-carbonyl)-4-(2-chloro-phenyl)-6-methyl-6H-pyrrolo[3,4-c]carbazole-1,3-dione

1. An isolated peptide that is defined by the three-dimensional atomic coordinates of the Wee1 peptide as set forth in Table 1, Table 2, Table 3, Table 4, or Table 5, or a related set of atomic coordinates having a root mean square deviation of not more than about 1.25 Å away from the core C alpha atoms of the three-dimensional atomic coordinates as set forth in Table 1, Table 2, Table 3, Table 4, or Table 5.

2. An isolated Wee1 peptide having
an NH₂-terminal truncation lacking at most about 290 amino acid residues from the NH₂-terminal region of the full-length Wee1 peptide, and
a COOH-truncation lacking at most about 70 amino acid residues from the COOH-terminal region of the full-length Wee1 peptide.

3. The Wee1 peptide of claim 2, wherein the peptide has an amino acid sequence from amino acid 291 to amino acid 575 of SEQ ID NO: 2 or a conservatively substituted variant thereof.

4. An isolated peptide comprising an ATP substrate binding site selected from the group consisting of:
(a) an ATP substrate binding site that is defined by the three-dimensional atomic coordinates of the following amino acid residues within about 5 Å of an inhibitor located in the ATP substrate binding site: I305, G306, V313, A326, K328, E346, V360, I374, N376, E377, Y378, C379, N380, G381, G382, S383, D386, N431, F433, G462, D463, L464, G465 of SEQ ID NO: 2, or a conservatively substituted variant thereof;
(b) an ATP substrate binding site defined by the following amino acid residues: I305, V313, A326, N376, E377, Y378, C379, G382, and F433 of SEQ ID NO: 2, or a conservatively substituted variant thereof;
(c) an ATP substrate binding site defined by the following amino acid residues: V313, K328, E346, D463, N376, and I374 of SEQ ID NO: 2, or a conservatively substituted variant thereof;
(d) an ATP substrate binding site defined by the following amino acid residues: V313, K328, E346, D463, N376, and I374 of SEQ ID NO: 2, or a conservatively substituted variant thereof, and a bound magnesium ion and its associated waters;
(e) an ATP substrate binding site defined by the following amino acid residues: V313, K328, E346, D463, N376, and I374 of SEQ ID NO: 2, or a conservatively substituted variant thereof, and tightly bound water molecules; and
(f) an ATP substrate binding site that is defined by the atoms found in the three-dimensional atomic coordinates of the Wee1 peptide as set forth in Table 1, Table 2, Table 3, Table 4, or Table 5, or in a related set of atomic coordinates having a root mean square deviation of not more than about 1.25 Å away from the binding site C alpha atoms of the ATP substrate binding site according to (a), (b), (c) or (d), or a conservatively substituted variant thereof.

5. A crystalline structure of a peptide: inhibitor complex, wherein the peptide is a Wee1 peptide having a NH₂-terminal truncation lacking at most about 290 amino acid residues from the NH₂-terminal region of a full-length Wee1 peptide and a COOH-terminal truncation lacking at most about 70 amino acid residues from the COOH-terminal region of the Wee1 peptide.

6. A crystalline structure of a peptide: inhibitor complex, wherein the peptide is defined by the three-dimensional atomic coordinates of the Wee1 peptide as set forth in Table 1, Table 2, Table 3, Table 4, or Table 5, or a related set of atomic coordinates having a root mean square deviation of not more than about 1.25 Å away from the core C alpha atoms of the three-dimensional atomic coordinates as set forth in Table 1, Table 2, Table 3, Table 4, or Table 5.

7. The crystalline structure of claim 5, wherein the Wee1 peptide has an amino acid sequence from amino acid 291 to amino acid 575 of SEQ ID NO: 2 or a conservatively substituted variant thereof.

8. The crystalline structure of claim 7, wherein the inhibitor is selected from the group consisting of:
9-Hydroxy-4-phenyl-6H-pyrrolo[3,4-c]carbazole-1,3-dione;
4-(2-Chloro-phenyl)-8-(3-dimethylamino-propoxy)-9-hydroxy-6-methyl-6H-pyrrolo[3,4-c]carbazole-1,3-dione;
3-(9-Hydroxy-1,3-dioxo-4-phenyl-2,3-dihydro-1 H-pyrrolo[3,4-c]carbazol-6-yl)-propionic acid;
9-Hydroxy-6-(3-hydroxy-propyl)-4-(2-methoxy-phenyl)-6H-pyrrolo[3,4-c]carbazole-1,3-dione; and
8-(3-Amino-pyrrolidine-1-carbonyl)-4-(2-chloro-phenyl)-6-methyl-6H-pyrrolo[3,4-c]carbazole-1,3-dione.

9. A crystalline structure of an isolated peptide comprising an ATP substrate binding site selected from the group consisting of:
(a) an ATP substrate binding site that is defined by the atomic coordinates of the following amino acid residues within about 5 Å of an inhibitor located in the ATP substrate binding site: I305, G306, V313, A326, K328, E346, V360, I374, N376, E377, Y378, C379, N380, G381, G382, S383, D386, N431, F433, G462, D463, L464, G465 of SEQ ID NO: 2, or a conservatively substituted variant thereof;
(b) an ATP substrate binding site defined by the following amino acid residues: I305, V313, A326, N376, E377, Y378, C379, G382, and F433 of SEQ ID NO: 2, or a conservatively substituted variant thereof;
(c) an ATP substrate binding site defined by the following amino acid residues: V313, K328, E346, D463, N376, and I374 of SEQ ID NO: 2, or a conservatively substituted variant thereof;
(d) an ATP substrate binding site defined by the following amino acid residues: V313, K328, E346, D463, N376, and I374 of SEQ ID NO: 2, or a conservatively substituted variant thereof, and a bound magnesium ion and its associated waters;
(e) an ATP substrate binding site defined by the following amino acid residues: V313, K328, E346, D463, N376, and I374 of SEQ ID NO: 2, or a conservatively substituted variant thereof, and tightly bound water molecules; and
(f) an ATP substrate binding site that is defined by the atoms found in the three-dimensional atomic coordinates of the Wee1 peptide as set forth in Table 1, Table 2, Table 3, Table 4, or Table 5, or in a related set of atomic coordinates having a root mean square deviation of not more than about 1.25 Å away from the binding site C alpha atoms of the ATP substrate binding site according to (a), (b), (c) or (d), or a conservatively substituted variant thereof.

10. The crystalline structure of claim 9, wherein the peptide is selected from the group consisting of:
a Wee1 peptide having a NH₂-terminal truncation lacking at most about 290 amino acid residues from the NH₂-terminal region of a full-length Wee1 peptide and a COOH-terminal truncation lacking at most about 70 amino acid residues from the COOH-terminal region of the Wee1 peptide; and
a peptide that is defined by the three-dimensional atomic coordinates of the Wee1 peptide as set forth in Table 1, Table 2, Table 3, Table 4, or Table 5, or a related set of atomic coordinates having a root mean square deviation of not more than about 1.25 Å away from the core C alpha atoms of the three-dimensional atomic coordinates as set forth in Table 1, Table 2, Table 3, Table 4, or Table 5.

11. Three-dimensional atomic coordinates of a peptide: inhibitor complex comprising:
a peptide selected from the group consisting of:
a Wee1 having a NH₂-terminal truncation lacking at most about 290 amino acid residues from the NH₂-terminal region of a full-length Wee1 peptide and a COOH-terminal truncation lacking at most about 70 amino acid residues from the COOH-terminal region of the Wee1 peptide; and
a peptide that is defined by the three-dimensional atomic coordinates of the Wee1 peptide as set forth in Table 1, Table 2, Table 3, Table 4, or Table 5, or a related set of atomic coordinates having a root mean square deviation of not more than about 1.25 Å away from the core C alpha atoms of the three-dimensional atomic coordinates as set forth in Table 1, Table 2, Table 3, Table 4, or Table 5; and
an inhibitor,
wherein the complex has the atomic coordinates set forth in any one of Tables 1-5.

12. The three-dimensional atomic coordinates of claim 11, wherein the inhibitor is selected from the group consisting of:
9-Hydroxy-4-phenyl-6H-pyrrolo[3,4-c]carbazole-1,3-dione;
4-(2-Chloro-phenyl)-8-(3-dimethylamino-propoxy)-9-hydroxy-6-methyl-6H-pyrrolo[3,4-c]carbazole-1,3-dione;
3-(9-Hydroxy-1,3-dioxo-4-phenyl-2,3-dihydro-1H-pyrrolo[3,4-c]carbazol-6-yl)-propionic acid;
9-Hydroxy-6-(3-hydroxy-propyl)-4-(2-methoxy-phenyl)-6H-pyrrolo[3,4-c]carbazole-1,3-dione; and
8-(3-Amino-pyrrolidine-1-carbonyl)-4-(2-chloro-phenyl)-6-methyl-6H-pyrrolo[3,4-c]carbazole-1,3-dione.

13. The three-dimensional atomic coordinates according to claim 12, wherein the Wee1 peptide has an amino acid sequence from amino acid 291 to amino acid 575 of SEQ ID NO: 2 or a conservatively substituted variant thereof.

14. An expression vector for producing the peptide according to claim 1 or 2 in a host cell comprising a polynucleotide encoding the peptide and transcriptional and translational regulatory sequences functional in the host cell operably linked to the peptide.

15. A host cell stably transformed and transfected with a polynucleotide selected from the group consisting of a polynucleotide encoding the peptide according to claim 1 or 2, or a conservatively substituted variant thereof.

16. A method of utilizing molecular replacement to obtain structural information about a molecule or a molecular complex of unknown structure comprising:
crystallizing said molecule or molecular complex;
generating an X-ray diffraction pattern from said crystallized molecule or molecular complex; and
applying at least a portion of the three-dimensional atomic coordinates set forth in any one of Tables 1-5 to the X-ray diffraction pattern to generate a three-dimensional electron density map of at least a portion of the molecule or molecular complex whose structure is unknown.

17. A machine-readable medium having stored thereon data comprising the three-dimensional atomic coordinates as set forth in Table 1, Table 2, Table 3, Table 4, or Table 5, or a related set of atomic coordinates having a root mean square deviation of not more than about 1.25 Å away from the core C alpha atoms of the three-dimensional atomic coordinates as set forth in Table 1, Table 2, Table 3, Table 4, or Table 5.

18. A method for generating a three-dimensional computer representation of a Wee1 peptide, a structurally related peptide, or an ATP substrate binding site, comprising applying the three-dimensional atomic coordinates set forth in Table 1, Table 2, Table 3, Table 4, or Table 5, or a related set of atomic coordinates having a root mean square deviation of not more than about 1.25 Å away from the core C alpha atoms of the three-dimensional atomic coordinates as set forth in Table 1, Table 2, Table 3, Table 4, or Table 5, to a computer algorithm to generate the three-dimensional representation,
wherein the ATP substrate binding site is selected from the group consisting of:
(a) an ATP substrate binding site that is defined by the atomic coordinates of the following amino acid residues within about 5 Å of an inhibitor located in the ATP substrate binding site: I305, G306, V313, A326, K328, E346, V360, I374, N376, E377, Y378, C379, N380, G381, G382, S383, D386, N431, F433, G462, D463, L464, G465 of SEQ ID NO: 2, or a conservatively substituted variant thereof;
(b) an ATP substrate binding site defined by the following amino acid residues: I305, V313, A326, N376, E377, Y378, C379, G382, and F433 of SEQ ID NO: 2, or a conservatively substituted variant thereof;
(c) an ATP substrate binding site defined by the following amino acid residues: V313, K328, E346, D463, N376, and I374 of SEQ ID NO: 2, or a conservatively substituted variant thereof;
(d) an ATP substrate binding site defined by the following amino acid residues: V313, K328, E346, D463, N376, and I374 of SEQ ID NO: 2, or a conservatively substituted variant thereof, and a bound magnesium ion and its associated waters;
(e) an ATP substrate binding site defined by the following amino acid residues: V313, K328, E346, D463, N376, and I374 of SEQ ID NO: 2, or a conservatively substituted variant thereof, and tightly bound water molecules; and
(f) an ATP substrate binding site that is defined by the atoms found in the three-dimensional atomic coordinates of the Wee1 peptide as set forth in Table 1, Table 2, Table 3, Table 4, or Table 5, or in a related set of atomic coordinates having a root mean square deviation of not more than about 1.25 Å away from the binding site C alpha atoms of the ATP substrate binding site according to (a), (b), (c) or (d), or a conservatively substituted variant thereof.

19. A method for modifying a chemical entity having the potential to associate with a Wee1 peptide or a structurally related peptide, comprising:
(a) generating a three-dimensional computer representation according to the method of claim 18;
(b) modeling the chemical entity based on said three-dimensional representation; and
(c) modifying the chemical entity to improve its ability to associate with the peptide or ATP substrate binding site.

20. The method according claim 19, wherein the modeling step (b) comprises:
(1) employing computational means to perform a fitting operation between the chemical entity and the peptide or ATP substrate binding site; and
(2) evaluating the results of said fitting operation to quantify the association between the chemical entity and the peptide or ATP substrate binding site.

21. The method according to claim 19, further comprising
(d) growing a crystal comprising the peptide and the modified chemical entity; and
(e) determining the three-dimensional structure of the crystal using molecular replacement.

22. A method for designing a chemical entity having the potential to associate with a Wee1 peptide or a structurally related peptide, comprising:
(a) generating a three-dimensional computer representation according to the method of claim 18;
(b) generating a chemical entity that spatially conforms to the three-dimensional representation of the peptide or a ATP substrate binding site of the peptide; and
(c) evaluating whether the chemical entity has the potential to associate with the peptide or ATP substrate binding site.

23. The method according to claim 22, wherein the chemical entity is generated by a method selected from the group consisting of (i) assembling molecular fragments into the chemical entity; (ii) de novo design of the chemical entity; (iii) selecting a chemical entity from a small molecule database; and (iv) modifying a known inhibitor, or portion thereof, of Wee1 activity.

24. A method for screening and identifying a potential inhibitor of the activity of a Wee1 peptide or a structurally related peptide, comprising:
(a) generating a three-dimensional representation according to the method of claim 18;
(b) applying an iterative process whereby a chemical entity is applied to the three-dimensional representation to determine whether the chemical entity associates with the peptide or ATP substrate binding site; and
(c) evaluating the effect(s) of the chemical entity on peptide activity to determine whether the chemical entity functions as an activity inhibitor.

25. The method of claim 24, wherein the iterative process comprises selecting a chemical entity to be evaluated by a method selected from the group consisting of (i) assembling molecular fragments into the compound; (ii) de novo design of the compound or fragment; (iii) selecting a compound from a small molecule database; and (iv) modifying a known inhibitor, or portion thereof, of Wee1 activity.

26. A method for screening and identifying a potential inhibitor of the activity of a Wee1 peptide or a structurally related peptide, comprising:
(a) generating a three-dimensional representation of an ATP substrate binding site selected from the group consisting of:
(1) an ATP substrate binding site that is defined by the atomic coordinates of the following amino acid residues within about 5 Å of an inhibitor located in the ATP substrate binding site: I305, G306, V313, A326, K328, E346, V360, I374, N376, E377, Y378, C379, N380, G381, G382, S383, D386, N431, F433, G462, D463, L464, G465 of SEQ ID NO: 2, or a conservatively substituted variant thereof;
(2) an ATP substrate binding site defined by the following amino acid residues: I305, V313, A326, N376, E377, Y378, C379, G382, and F433 of SEQ ID NO: 2, or a conservatively substituted variant thereof;
(3) an ATP substrate binding site defined by the following amino acid residues: V313, K328, E346, D463, N376, and I374 of SEQ ID NO: 2, or a conservatively substituted variant thereof;
(4) an ATP substrate binding site defined by the following amino acid residues: V313, K328, E346, D463, N376, and I374 of SEQ ID NO: 2, or a conservatively substituted variant thereof, and a bound magnesium ion and its associated waters;
(5) an ATP substrate binding site defined by the following amino acid residues: V313, K328, E346, D463, N376, and I374 of SEQ ID NO: 2, or a conservatively substituted variant thereof, and tightly bound water molecules; and
(6) an ATP substrate binding site that is defined by the atoms found in the three-dimensional atomic coordinates of the Wee1 peptide as set forth in Table 1, Table 2, Table 3, Table 4, or Table 5, or in a related set of atomic coordinates having a root mean square deviation of not more than about 1.25 Å away from the binding site C alpha atoms of the ATP substrate binding site according to (a)(1), (a)(2), (a)(3), (a)(4) or (a)(5), or a conservatively substituted variant thereof,
by applying the three-dimensional atomic coordinates set forth in Table 1, Table 2, Table 3, Table 4, or Table 5, or a related set of atomic coordinates having a root mean square deviation of not more than about 1.25 Å from the core C alpha atoms of the three-dimensional atomic coordinates set forth in Table 1, Table 2, Table 3, Table 4, or Table 5, to a computer algorithm to generate a three-dimensional representation of the ATP substrate binding site;
(b) generating a potential inhibitor by (i) assembling molecular fragments into a chemical entity; (ii) de novo design of a chemical entity; (iii) selecting a chemical entity from a small molecule database; or (iv) modifying a known chemical entity; and
(c) evaluating by computer modeling whether the potential inhibitor associates with the ATP substrate binding site.

27. The method according to claim 26, further comprising
(d) modifying the known chemical entity to improve its ability to associate with the ATP substrate binding site.

28. A method for screening and identifying a potential inhibitor of the activity of a Wee1 peptide or a structurally related peptide, comprising:
(a) generating a three-dimensional representation of an ATP substrate binding site selected from the group consisting of:
(1) an ATP substrate binding site that is defined by the atomic coordinates of the following amino acid residues within about 5 Å of an inhibitor located in the ATP substrate binding site: I305, G306, V313, A326, K328, E346, V360, I374, N376, E377, Y378, C379, N380, G381, G382, S383, D386, N431, F433, G462, D463, L464, G465 of SEQ ID NO: 2, or a conservatively substituted variant thereof;
(2) an ATP substrate binding site defined by the following amino acid residues: I305, V313, A326, N376, E377, Y378, C379, G382, and F433 of SEQ ID NO: 2, or a conservatively substituted variant thereof;
(3) an ATP substrate binding site defined by the following amino acid residues: V313, K328, E346, D463, N376, and I374 of SEQ ID NO: 2, or a conservatively substituted variant thereof;
(4) an ATP substrate binding site defined by the following amino acid residues: V313, K328, E346, D463, N376, and I374 of SEQ ID NO: 2, or a conservatively substituted variant thereof, and a bound magnesium ion and its associated waters;
(5) an ATP substrate binding site defined by the following amino acid residues: V313, K328, E346, D463, N376, and I374 of SEQ ID NO: 2, or a conservatively substituted variant thereof, and tightly bound water molecules; and
(6) an ATP substrate binding site that is defined by the atoms found in the three-dimensional atomic coordinates of the Wee1 peptide as set forth in Table 1, Table 2, Table 3, Table 4, or Table 5, or in a related set of atomic coordinates having a root mean square deviation of not more than about 1.25 Å away from the binding site C alpha atoms of the ATP substrate binding site according to (a)(1), (a)(2), (a)(3), (a)(4) or (a)(5), or a conservatively substituted variant thereof,
by applying the three-dimensional atomic coordinates set forth in Table 1, Table 2, Table 3, Table 4, or Table 5, or a related set of atomic coordinates having a root mean square deviation of not more than about 1.25 Å from the core C alpha atoms of the three-dimensional atomic coordinates set forth in Table 1, Table 2, Table 3, Table 4, or Table 5, to a computer algorithm to generate a three-dimensional representation of the ATP substrate binding site;
(b) generating a chemical entity that spatially conforms to the ATP substrate binding site, wherein the chemical entity is generated by (i) assembling molecular fragments into the chemical entity; (ii) de novo design of the chemical entity; (iii) selecting the chemical entity from a small molecule database; or (iv) modifying a known inhibitor, or portion thereof, of Wee1 activity;
(c) synthesizing the chemical entity or analogs thereof; and
(d) evaluating whether the chemical entity associates with the ATP substrate binding site.

29. The method according to claim 28, further comprising
(e) growing a crystal comprising the peptide and the chemical entity; and
(f) determining the three-dimensional structure of the crystal using molecular replacement.

30. A method for evaluating the potential of a chemical entity to associate with a Wee1 peptide or a structurally related peptide, comprising:
(a) generating a three dimensional representation according to the method of claim 18;
(b) applying a three dimensional representation of the chemical entity to the three-dimensional representation generated according to the method of claim 18; and
(c) quantifying the association between the chemical entity and the peptide or ATP substrate binding site.

31. A method for evaluating the potential of a chemical entity to associate with a Wee1 peptide or a structurally related peptide, comprising
(a) generating a three-dimensional representation of an ATP substrate binding site selected from the group consisting of:
(1) an ATP substrate binding site that is defined by the atomic coordinates of the following amino acid residues within about 5 Å of an inhibitor located in the ATP substrate binding site: I305, G306, V313, A326, K328, E346, V360, I374, N376, E377, Y378, C379, N380, G381, G382, S383, D386, N431, F433, G462, D463, L464, G465 of SEQ ID NO: 2, or a conservatively substituted variant thereof;
(2) an ATP substrate binding site defined by the following amino acid residues: I305, V313, A326, N376, E377, Y378, C379, G382, and F433 of SEQ ID NO: 2, or a conservatively substituted variant thereof;
(3) an ATP substrate binding site defined by the following amino acid residues: V313, K328, E346, D463, N376, and I374 of SEQ ID NO: 2, or a conservatively substituted variant thereof;
(4) an ATP substrate binding site defined by the following amino acid residues: V313, K328, E346, D463, N376, and I374 of SEQ ID NO: 2, or a conservatively substituted variant thereof, and a bound magnesium ion and its associated waters;
(5) an ATP substrate binding site defined by the following amino acid residues: V313, K328, E346, D463, N376, and I374 of SEQ ID NO: 2, or a conservatively substituted variant thereof, and tightly bound water molecules; and
(6) an ATP substrate binding site that is defined by the atoms found in the three-dimensional atomic coordinates of the Wee1 peptide as set forth in Table 1, Table 2, Table 3, Table 4, or Table 5, or in a related set of atomic coordinates having a root mean square deviation of not more than about 1.25 Å away from the binding site C alpha atoms of the ATP substrate binding site according to (a)(1), (a)(2), (a)(3), (a)(4) or (a)(5), or a conservatively substituted variant thereof,
by applying the three-dimensional atomic coordinates set forth in Table 1, Table 2, Table 3, Table 4, or Table 5, or a related set of atomic coordinates having a root mean square deviation of not more than about 1.25 Å from the core C alpha atoms of the three-dimensional atomic coordinates set forth in Table 1, Table 2, Table 3, Table 4, or Table 5, to a computer algorithm to generate a three-dimensional representation of the ATP substrate binding site;
(b) applying a chemical entity to the three-dimensional representation; and
(c) quantifying the association between the chemical entity and the ATP substrate binding site.

32. The method of claim 30 or claim 31, wherein the association is quantified by:
(1) employing computational means to perform a fitting operation between the chemical entity and the computer representation of the peptide or ATP substrate binding site; and
(2) analyzing the results of said fitting operation to determine the association between the chemical entity and the computer representation of the peptide or ATP substrate binding site.

33. A method of purifying a Wee1 peptide from a cell culture containing the peptide and contaminant proteins other than the peptide comprising
subjecting the cell culture to mechanical lysis and cobalt metal affinity chromatography;
cleaving a histidine tag;
performing dialysis; and
subjecting the resulting solution to size exclusion chromatograph,
wherein the Wee1 peptide has an NH₂-terminal truncation lacking at most about 290 amino acid residues from the NH₂-terminal region of the full-length Wee1 peptide, and a COOH-truncation lacking at most about 70 amino acid residues from the COOH-terminal region of the full-length Wee1 peptide.

34. A method for producing crystalline complexes of a Wee1 peptide and an inhibitor, comprising
contacting a purified Wee1 peptide with an inhibitor in the presence of one or more of a buffering agent, a reducing agent, a source of ionic strength, an organic agent, and a metal cation chelating agent to form a Wee1: inhibitor binary complex solution;
adding the solution of the binary complex to a crystallization solution comprising at least one source of ionic strength and a buffering agent, to form a Wee 1 peptide: inhibitor crystal, wherein the Wee1 peptide has an NH₂-terminal truncation lacking at most about 290 amino acid residues from the NH₂-terminal region of the full-length Wee1 peptide, and a COOH-truncation lacking at most about 70 amino acid residues from the COOH-terminal region of the full-length Wee1 peptide.
